(19) United States
(12) Patent Application Publication
(10) Pub. No.: US 2016/0362463 A1
(43) Pub. Date: Dec. 15, 2016

(54) Pharmaceutical composition inhibiting interaction between MZF-1 and Elk-1

(71) Applicant: China Medical University, Taichung City (TW)

(72) Inventors: Jer-Yuh Liu, Taichung City (TW); Chia-Jan Lee, Taichung City (TW)

(21) Appl. No.: 14/739,772

(22) Filed: Jun. 15, 2015

(51) Int. Cl. C07K 14/47

(52) U.S. Cl. CPC C07K 14/4702; A61K 38/00

(57) ABSTRACT

This invention discloses a peptide, which inhibits the interaction between of MZF-1 and Elk-1 and further inhibits cancers. Both myeloid zinc finger 1 (MZF-1) and Ets-like protein-1 (Elk-1) expressions correlate to PKCα expression in cancer cells. Furthermore, it is the interaction between the acidic domain of MZF-1 and the heparin-binding domain of Elk-1 which facilitated their heterodimeric complex formation before their binding to the PKCα promoter. Blocking the formation of the heterodimer changed Elk-1 nuclear localization, MZF-1 protein degradation, their DNA-binding activities, and subsequently the expression of PKCα in cancer cells. Thus, migration, tumorigenicity, and epithelial-mesenchymal transition potential of cancer cells decreased, suggesting that the Elk-1/MZF-1 heterodimer is considered as a mediator of PKCα in TNBC cell malignancy. The obtained data also suggest that the next therapeutic strategy in the treatment of cancer will come from the blocking of Elk-1/MZF-1 interaction through the saturation of Elk-1 or MZF-1 binding domains, such as through the application of cell-penetrating HIV transactivating regulatory protein-fused peptides.

### BACKGROUND OF THE INVENTION

1. Field of the Invention

The present disclosure relates to a peptide, particularly a peptide interrupting interaction between MZF-1 and Elk-1 and inhibiting cancers. The present disclosure further relates to a method for treating of a patient having a cancer, particularly a method disrupting the interaction between MZF-1 and Elk-1 and the inhibiting cancers.

2. Description of the Prior Art

Elk-1 (Ets-like protein-1) is a transcription factor as a member of the ternary complex factor (TCF) subfamily of Ets domain proteins. TCFs are able to form a ternary complex with the serum response factor (SRF) and the serum-response element (SRE). As a subgroup of TCFs, Ets protein family members, Elk-1, Sap1, and Sap2, possess an Ets domain and a winged helix-loop-helix (HLH) DNA binding domain recognizing specific DNA sequences. It has been found that the N-terminal Ets-DNA binding domain of Elk-1 is important for DNA recognition. TCFs contain a “B box” domain containing 20 amino acids which mediates protein-protein interaction; C-terminal of Elk-1 possesses phosphorylation sites for mitogen-activated protein kinase (MAPK); D and F×FP domains that act as docking site for their interaction with activates MAPK. Elk-1 specifically targets genes that encode proteins that are important in cell migration, cellular metastasis, and associate with the actin cytoskeleton.

MZF-1 (myeloid zinc finger-1) is a transcription factor of the Kruppel family of zinc finger proteins which contains three isoforms MZF-1, MZF-1B, and MZF-1C, MZF-1 is usually present in hematopoietic progenitor cells of myeloid lineage. Overexpression of MZF-1 induces migratory, invasive and in vivo metastatic potential of solid tumor cells. MZF-1 plays crucial roles in regulating normal haemopoiesis. However, the biological function of MZF-1 is not clear yet.

Breast cancer is the most common cancer in women. In 2011, 220,097 women were diagnosed with breast cancer and 40,931 women died from breast cancer in the United States. Triple-negative breast cancer (TNBC) make up most of the breast cancer phenotype that remains difficult to treat due to them being negative for estrogen receptor (ER), progesterone receptor (PR), or HER2 expression. Treatment for TNBC currently continues to involve conventional chemotherapy but relapse leading to a worse outcome happens frequently due to high metastasis rates and the lack of effective treatment. It is proposed that the presence of cancer stem cells (CSCs) or tumor-initiating cells (TICs) can account for treatment failure and TNBC recurrence, and breast TICs (BTICs) were later confirmed to be capable of reinitiating tumor growth after treatment and are responsible for tumor initiation, progression, and drug resistance. However, although diagnosis is becoming more accurate through the identification of TNBC/BTICs which allows for specific molecular targeting, clinical trials have yet to produce beneficial results.

Protein kinase C alpha (PKCα) is a central regulatory node in populations of cells with breast CSCs. PKCα express in both TNBC/BTICs cell lines and tumor samples correlated with poorer survival outcomes. These findings add TNBC to the list of cancers to which PKCα may be employed as a unique prognostic marker and an achievable therapeutic target. Several mechanisms that contribute to PKCα expression have been investigated. These mechanisms include the shift in signaling from epidermal growth factor receptor to platelet-derived growth factor receptor during progression from non-stem cells to cancer stem cells and epithelial-mesenchymal transition (EMT).

Up to now, the inhibitors of PKCα tested include chemical compounds (Riluzole), antisense oligonucleotide (Aprinocarsen) and peptide inhibitor (αV5-3). All are identifiable on the inhibition of PKCα activity and did not specifically target the cancer. The present invention characterizes a peptide targeting on only cancer cells to modulate PKCα expression and inhibiting cancer cells.

### SUMMARY OF THE INVENTION

One aspect of the invention is to provide a pharmaceutical composition for inhibiting interaction between MZF-1 and Elk-1, wherein said pharmaceutical composition comprises of at least one selected from the group consisting of:
(A) Peptides of MZF-1₆₀₋₇₂ having at least 50% sequence identity thereto SEQ ID NO:65, with variations except in the 1st, 3rd, 6th, and 12th amino acid (Aspartic acid, Asp, D);
(B) Peptides of Elk-1₁₄₅₋₁₅₇ having at least 50% sequence identity thereto SEQ ID NO:66, with variations except in the 3rd, 6th, and 11th amino acid (Arginine, Arg, R);
(C) TAT-fused peptide MZF-1₆₀₋₇₂; and
(D) TAT-fused peptide Elk-1₁₄₅₋₁₅₇.

According to the invention, the TAT-fused peptide MZF-1₆₀₋₇₂ sequence is set forth in SEQ ID NO: 57.

According to the invention, the TAT-fused peptide Elk-1₁₄₅₋₁₅₇ sequence is set forth in SEQ ID NO: 59.

According to the invention, the pharmaceutical composition further comprises of pharmaceutically acceptable vehicles, wherein said vehicles include excipients, diluents, thickeners, fillers, binders, disintegrants, lubricants, oil or non-oil agents, surfactants, suspending agents, gelling agents, adjuvants, preservatives, antioxidants, stabilizers, coloring agents or spices thereof.

According to the invention, the pharmaceutical composition is given by oral administration, immersion, injection, topical application or patch administration.

Another aspect of the invention is to provide a method for treatment to a patient having a cancer, said method comprises of administering to the patient a therapeutically effective amount of at least one selected from the group consisting of:
(A) Peptides of MZF-1₆₀₋₇₂, the sequence as set forth in SEQ ID NO:65;
(B) Peptide Elk-1₁₄₅₋₁₅₇, the sequence as set forth in SEQ ID NO:66;
(C) Peptides of MZF-1₆₀₋₇₂ having at least 50% sequence identity thereto SEQ ID NO:65, with variations except in the 1st, 3rd, 6th, and 12th amino acid (Aspartic acid, Asp, D);
(D) Peptides of Elk-1₁₄₅₋₁₅₇ having at least 50% sequence identity thereto SEQ ID NO:66, with variations except in the 3rd, 6th, and 11th amino acid (Arginine, Arg, R);
(E) TAT-fused peptide MZF-1₆₀₋₇₂; and
(F) TAT-fused peptide Elk-1₁₄₅₋₁₅₇;

According to the invention, the TAT-fused peptide MZF-1₆₀₋₇₂ sequence as set forth in SEQ ID NO: 57.

According to the invention, the TAT-fused peptide Elk-1₁₄₅₋₁₅₇ sequence as set forth in SEQ ID NO: 59.

According to the invention, the method inhibits interaction between MZF-1 and Elk-1.

According to the invention, the method reduces tumor volume.

According to the invention, the cancer more preferably breast cancers, liver cancers or cancers expressing interaction between MZF-1 and Elk-1.

According to the invention, the breast cancer is triple-negative breast cancer.

### BRIEF DESCRIPTION OF THE DRAWINGS

FIG. 1 shows immunohistochemical analyses and correlations of PKCα and Elk-1/MZF-1 expression in human breast cancer.

FIG. 2 shows immunohistochemical analyses and correlations of PKCα and Elk-1/MZF-1 expression in human liver cancer.

FIG. 3 shows immunohistochemical analyses and correlations of PKCα and Elk-1/MZF-1 expression in human lung cancer.

FIG. 4 shows immunohistochemical analyses and correlations of PKCα and Elk-1/MZF-1 expression in human bladder cancer.

FIG. 5 shows immunohistochemical analyses and correlations of PKCα and Elk-1/MZF-1 expression in human TNBC. Transcription activity compare with luciferase activity of TNBC cell line co-transfected with 2.5 μg MZF-1 or Elk-1 or MZF-1/Elk-1 expression vector and control cells.

FIG. 6 shows luciferase activity of two Hepatocellular carcinoma (HCC) cell lines co-transfect with the p[-660/-600]PKC α promoter luciferase plasmid, a β-galactosidase plasmid and either an empty vector or the indicated expression plasmids.

FIG. 7 shows the MZF-1/Elk-1 binding sequence at the PKCα promoter in which one the wild-type (WT) and three of the mutated sequences were designed.

FIG. 8 shows Electrophoretic Mobility Shift Assay (EMSA) analyses of specific binding activities of MZF-1/Elk-1 at the PKCα promoter in DMA-MB-231 cells. Samples were recognized by anti-Elk-1 antibody and anti-MZF-1 antibody.

FIG. 9 shows EMSA analyses of Verification of the specific binding activities of Elk-1/MZF-1 by a competitive assay in DMA-MB-231 cells. Samples were recognized by anti-Elk-1 antibody and anti-MZF-1 antibody, and competition EMSA of biotin-labeled oligonucleotides with unlabeled wild type or mutated oligonucleotides in 20- to 100-fold molar excess

FIG. 10 shows Co-Immunoprecipitation analyses of interaction between endogenous MZF-1 and Elk-1. Samples were Immunoprecipitation analyses with anti-Elk-1 antibody and anti-MZF-1 antibody. After Immunoprecipitation, samples were Immunoblotting analyses with anti-Elk-1 antibody and anti-MZF-1 antibody.

FIG. 11 shows Co-Immunoprecipitation analyses of interaction between endogenous MZF-1 and Elk-1 in DMA-MB-231 cells. Cells transfected with 5 μg Elk-1-c-Myc-ΔDBD or FLAG-MZF-1-ΔDBD. Samples were Immunoprecipitation analyses with anti-Elk-1 antibody and anti-MZF-1 antibody. After Immunoprecipitation, samples were Immunoblotting analyses with anti-Elk-1 antibody and anti-MZF-1 antibody.

FIG. 12 shows Confirmation of interactions between MZF-1 and Elk-1 and DNA binding activity by chromatin immunoprecipitation (ChIP) and Re-ChIP assays. In the ChIP assay, chromatin was pulled down with anti-Elk-1 antibody and anti-MZF-1 antibody. In the Re-ChIP assay, the pulled down chromatin was incubated with anti-Elk-1 antibodies, followed by anti-MZF-1 antibodies; the sequence was then reversed. The band indicated by an arrow corresponds to amplification of the PRKCA promoter by PCR performed.

FIG. 13 shows Antisense oligonucleotide-mediated gene knockdown assay was performed to determine the requirement for heterodimer formation. The ChIP and Re-ChIP assays was performed on SK-Hep-1 cells, which were transfected with 5 μM of sense or antisense MZF-1 oligonucleotide (S-MZF-1 or AS-MZF-1), or sense or antisense Elk-1 oligonucleotide (S-Elk-1 or AS-Elk-1).

FIG. 14 shows MZF-1-c-Myc and its fragments (amino acids 73-485, 1-60, 1-72, 1-141 and 60-72) were used to pinpoint the binding domain of MZF-1 (upper panel). Red bands indicate the fragments that bound Elk-1 as present in the co-immunoprecipitation assay (lower panel). HEK-293 cells were transfected with 5 μg FLAG-Elk-1 and either empty vector or the indicated MZF-1-c-Myc fragment. The c-Myc-fused peptides were Immunoprecipitation by c-Myc monoclonal antibody and then Immunoblotting with the anti-FLAG antibodies. Lysates of HEK-293 cells transfected with the FLAG-Elk-1 only were also Immunoblotting as a control (Input).

FIG. 15 shows the two mutant fragments (amino acids 1-72 and 60-72) of MZF-1 in which the negatively charged aspartates in their binding domains were mutated to uncharged alanines.

FIG. 16 shows EMSA analyses of effect of normal and mutant fragments of MZF-1 on specific binding activities of Elk-1 and MZF-1 at the PKCα promoter. The biotin-labeled WT oligonucleotide probes were incubated with the indicated concentration of nuclear extracts of the different-treated HEK-293 cells, which were transfected with empty vector, Elk-1, MZF-1, MZF-160-72 or mutant MZF-160-72 and the reactions were resolved on a nondenaturing polyacrylamide gel. DNA-protein complexes are indicated by the black arrow-head.

FIG. 17 shows Elk-1-c-Myc and its fragments (amino acids 1-428, 1-86, 87-144, 87-325, 87-4281-86, 87-144, 87-325, 87-428 and 60-72) of Elk-1 used to pinpoint the binding domain (upper panel). Red bands indicate the fragments that bound MZF-1, as present in the co-immunoprecipitation assay (lower panel). HEK-293 cells were co-transfected with 5 μg of FLAG-MZF-1 construct and either empty vector or the indicated Elk-1-c-Myc fragment. Lysates of HEK-293 cells transfected with the FLAG-MZF-1 construct were also Immunoblotting as a control (Input).

FIG. 18 shows the two mutant Elk-1 fragments (amino acids 145-157 and 145-428) in which the negatively charged arginines in their binding domains were mutated to uncharged alanines.

FIG. 19 shows EMSA analyses of the effects of normal and mutant Elk-1 fragments on specific binding activities of Elk-1 and MZF-1 at the PKCα promoter. The biotin-labeled wild-type oligonucleotide probes were incubated with the indicated concentration of nuclear extracts of the different-treated HEK-293 cells, which were transfected with empty vector, Elk-1, MZF-1, Elk-1145-157 or mutant Elk-1145-157.

FIG. 20 shows changes in morphology and gene expression in MZF-160-72 construct-transfected stably cloned MDA-MB-231 cells. β-actin was used as a control, and c-Myc was used as a marker of transfected cells.

FIG. 21 shows Confocal microscopy showing the distribution of the Elk-1 and MZF-1 proteins. Cells were stained with antibodies against Elk-1 and MZF-1 followed by the appropriate FITC- or rhodamine-conjugated secondary antibodies. Confocal slices of 0.5 and 0.6 μm were obtained, and images were taken through the center of the nucleus. “N” indicates the nucleus; “C” indicates the cytosol.

FIG. 22 shows disrupting MZF-1/Elk-1 heterodimer formation by different inhibitors in Hs578T cells. Changes in protein levels in the parental and Elk-1145-157-transfected stable Hs578T cells as detected by immunoblotting analysis. Low-passage cells were seeded at a density of 3×105 cells in 60 mm tissue culture dishes and then transfected with the Elk-1145-157 construct (5 μg) using Lipofectamine 2000. After transfection for 6 hours the cells were washed three times in serum-free MEM and allowed to recover for 24 hours in fresh medium. Stable clones were selected with geneticin (G418; 600 μg/ml) at 37° C. for four weeks.

FIG. 23 shows Disrupting MZF-1/Elk-1 heterodimer formation by different inhibitors in HCC SK-Hep-1 cells. Changes in protein levels in the parental and Elk-1145-157-transfected stable HCC SK-Hep-1 cells as detected by immunoblotting analysis. Low-passage cells were seeded at a density of 3×105 cells in 60 mm tissue culture dishes and then transfected with the Elk-1145-157 construct (5 μg) using Lipofectamine 2000. After transfection for 6 hours the cells were washed three times in serum-free MEM and allowed to recover for 24 hours in fresh medium. Stable clones were selected with geneticin (G418; 600 μg/ml) at 37° C. for four weeks.

FIG. 24 shows ChIP assays indicating the binding activity of endogenous Elk-1 and MZF-1 to the PKCα promoter in various cells. The assays were performed using an anti-MZF-1 antibody (upper panel) or anti-Elk-1 antibody (lower panel).

FIG. 25 shows immunoblotting analyses of effects of the anti-cancer drugs acriflavine and cisplatin on the viability of various MDA-MB-231 cells. The level of multidrug resistance protein permeability glycoprotein (P-gp) was determined by upper panel. Cell growth was determined using the MTT assay 2 days after treatment (lower panel). The data indicate the mean±S.D. (n=3 in each group). **p<0.01 compared with parental cells.

FIG. 26 shows visualization and quantification of cell migration and proliferation of modified MDA-MB-231 cells.

FIG. 27 shows tumor growth in nude mice after xenografts of modified MDA-MB-231 and Hs578T cells (left panels). Tumors removed from the mice were weighed (right images and graph) and sliced before histological examination (bottom images). **p<0.01 compared with the empty vector-transfected MDA-MB-231 group, ##p<0.01 compared with the empty vector-transfected Hs578T group.

FIG. 28 shows the gene expression profiles of upregulated (left panel) and downregulated (right panel) EMT-related genes in MZF-160-72-transfected Hs578T and MDA-MB-231 cloned cells as determined by microarray with those of the parental cells.

FIG. 29 shows immunoblotting analysis of changes in protein levels in the parental and transfected cells and in the less malignant MDA-MB-468 (MB-468) and MCF-7 cells.

FIG. 30 shows visualization and quantification of cell migration of PKCα-transfected cells by migration assay. MZF-160-72 construct-transfected stably cloned cells were transfected with the empty-vector or full-length PKCα construct for 3 days and the migration assay was performed. **p<0.01 compared with the parental cells, ##p<0.01 compared with the empty vector-transfected groups.

FIG. 31 shows Immunoblotting analysis of changes in protein levels in the PKCα-co-transfected cells. β-actin was used as a control.

FIG. 32 shows sequences of the HIV trans-activating regulatory protein (TAT)-fused TAT-MZF-160-72 and TAT-Elk-1145-157 peptides (normal and mutant).

FIG. 33 shows effects of the TAT-fused peptides on cell migration. The cell migration assay was performed 3 days after the addition of the peptides to the cells.

FIG. 34 shows immunoblotting analysis of changes in protein levels in the TAT-fused peptide-treated cells three days post-treatment. β-actin was used as a control.

FIG. 35 shows co-immunoprecipitation detection of the effects of the TAT-fused peptides on the Elk-1 and MZF-1 interaction by. HEK-293 cells were transfected with empty vector, FLAG-Elk-1, MZF-1-c-Myc, FLAG-MZF-1 or Elk-1-cMyc construct. ‘+’ indicates the presence of each item and ‘-’ indicates their absence. Different concentrations (50, 100 and 150 nmol) of TAT-fused peptides (normal or mutant) were then added to the lysates and incubated overnight at 4° C. and then subjected to immunoprecipitation with anti-c-Myc antibody, followed by immunoblotting against FLAG and c-Myc. Lysates of HEK-293 cells transfected with FLAG-Elk-1 or FLAG-MZF-1 vector indicated as a “Input”.

FIG. 36 shows a scheme depicting the regulation of PKCα expression by the interaction of Elk-1 and MZF-1

### DETAILED DESCRIPTION OF THE PREFERRED EMBODIMENT

Unless defined otherwise all technical and scientific terms used herein have the meaning commonly understood by a person skilled in the art to which this invention belongs. As used herein, the following terms have the meanings ascribed more specifically with reference to the following embodiments, which are provided for the purpose of demonstration rather than limitation.

Present invention first reveals the correlation the interaction between MZF-1 and Elk-1 in cancer cells. Additional MZF-1 or Elk-1 fragment can block MZF-1/Elk-1 interaction, regulate PKCα expression and even inhibit cancer cells.

Plasmid Construction

The expression vectors described below were driven by the cytomegalovirus (CMV) promoter-basic contained in the pcDNA3 vector (Invitrogen). Open reading frames of the human MZF-1 (GenBank Accession No. AF161886 10781-12235 bp) and Elk-1 (GenBank Accession No. AB016193 101-1384 bp) genes were amplified from SK-Hep-1 cells by reverse transcription-polymerase chain reaction (RT-PCR) and cloned into vectors; the resulting recombinant plasmids were designated as pcDNA-MZF-1 and pcDNA-Elk-1, respectively. Table 1 lists the primer sequences and the restriction sites used for cloning. The PCR products were isolated and cloned into the pcDNA™ 3.1/myc-His vector (Invitrogen).

TABLE 1
Primers and restriction enzyme (R.E.) sites for the full-length and truncated MZF-1, Elk-1
and PKCα constructs.

| | | | | SEQ | | | SEQ | |
|---|---|---|---|---|---|---|---|---|
| | | | | ID | | | ID | |
| No. | constructs | A.A. | Primer forward | NO: | R.E. | Primer reverse | NO: | R.E. |
| 1 | MZF-1-c-Myc | 1-485 | GGAATTCCAATGAATGGTCCCCTTG | 1 | EcoRI | GGGGTACCCTCGGCGCTGTGGACG | 2 | KpnI |
| | | | TG | | | CG | | |
| 2 | MZF-1-c-Myc | 1-60 | GGAATTCCAATGAATGGTCCCCTTG | 3 | EcoRI | CCCAAGCTTGGGGCTGGGGAGCAG | 4 | HindIII |
| | | | TG | | | AACGCG | | |
| 3 | MZF-1-c-Myc | 1-72 | GGAATTCCAATGAATGGTCCCCTTG | 5 | EcoRI | GGGGTACCCCCATCCTCGTCCGTGG | 6 | KpnI |
| | | | TG | | | GGTC | | |
| 4 | MZF-1-c-Myc | 1-141 | GGAATTCCAATGAATGGTCCCCTTG | 7 | EcoRI | GGGGTACCCCCGCCGCACTCGCTGC | 8 | KpnI |
| | | | TG | | | AC | | |
| 5 | MZF-1-c-Myc | 73-485 | GGAATTCCACCCTGCCGGGGTGTGG | 9 | EcoRI | GGGGTACCCCCCTCGGCGCTGTGGA | 10 | KpnI |
| | | | GCCCT | | | CGCG | | |
| 6 | MZF-1-c-Myc | 60-72 | GGAATTCCAAGCGATCTGAGGAGT | 11 | EcoRI | CCCAAGCTTGGGATCCTCGTCCGTG | 12 | HindIII |
| | | | GAACAGGACCCCACGGACGAGGAT | | | GGGTCCTGTTCACTCCTCAGATCGCT | | |
| | | | CCCAAGCTTGGG | | | TGGAATTCC | | |
| 7 | MZF-1-c-Myc | 60-72 | GGAATTCCAAGCGCACTGAGGAGT | 13 | EcoRI | CCCAAGCTTGGGTGCCTCTGCCGTG | 14 | HindII |
| | | (Mutant) | GAACAGGCACCCACGGCAGAGGCA | | | GGTGCCTGTTCACTCCTCAGTGCGCT | | |
| | | | CCCAAGCTTGGG | | | TGGAATTCC | | |
| 8 | MZF-1-c-Myc | 1-72 | GGAATTCCAATGAATGGTCCCCTTG | 15 | EcoRI | CCCAAGCTTGGGTGCCTCTGCCGTG | 16 | HindIII |
| | | (Mutant) | TG | | | GGTGCCTGTTCACTCCTCAGTGCGCT | | |
| 9 | FLAG-MZF-1 | 1-485 | GGAATTCAATGAATGGTCCCCTTGT | 17 | EcoRI | CCGCTCGAGCTCGGCGCTGTGGACG | 18 | XhoI |
| | | | G | | | CG | | |
| 10 | FLAG-MZF-1 | 1-72 | GGAATTCAATGAATGGTCCCCTTGT | 19 | EcoRI | CCGCTCGAGCCCATCCTCGTCCGTG | 20 | XhoI |
| | | | G | | | GGGTC | | |
| 11 | Elk-1-c-Myc | 1-428 | CGGGATCCATGGACCCATCTGTGAC | 21 | BamHI | CCCAAGCTTTGGCTTCTGGGGCCCT | 22 | HindIII |
| | | | G | | | GG | | |
| 12 | Elk-1-c-Myc | 1-86 | CGGGATCCATGGACCCATCTGTGAC | 23 | BamHI | CCCAAGCTTCACAAACTTGTAGACG | 24 | HindIII |
| | | | G | | | AA | | |
| 13 | Elk-1-c-Myc | 87-144 | GGAATTCCATCCTACCCTGAGGTCG | 25 | EcoRI | CCCAAGCTTACCGCCTGGGCCTGCC | 26 | HindIII |
| | | | CT | | | AT | | |
| 14 | Elk-1-c-Myc | 87-325 | GGAATTCCATCCTACCCTGAGGTCG | 27 | EcoRI | CCCAAGCTTCGGGCTGAGTGGAAGC | 28 | HindIII |
| | | | CT | | | TC | | |
| 15 | Elk-1-c-Myc | 87-428 | GGAATTCCATCCTACCCTGAGGTCG | 29 | EcoRI | CCCAAGCTTTGGCTTCTGGGGCCCT | 30 | HindIII |
| | | | CT | | | GG | | |
| 16 | Elk-1-c-Myc | 321-428 | GGAATTCCACTTCCACTCAGCCCGA | 31 | EcoRI | CCCAAGCTTTGGCTTCTGGGGCCCT | 32 | HindIII |
| | | | GC | | | GG | | |
| 17 | Elk-1-c-Myc | 145-428 | CGGGATCCTTGGCACGCAGCAGCCG | 33 | BamHI | CCCAAGCTTTGGCTTCTGGGGCCCT | 34 | HindIII |
| | | | G | | | GG | | |
| 18 | Elk-1-c-Myc | 145-157 | GGAATTCCATTGGCACGCAGCAGCC | 35 | EcoRI | CCCAAGCTTGCCCGAGCGCATGTAC | 36 | HindIII |
| | | | GGAACGAGTACATGCGCTCGGGCA | | | TCGTTCCGGCTGCTGCGTGCCAATG | | |
| | | | AGCTTGGG | | | GAATTCC | | |
| 19 | Elk-1-c-Myc | 309-320 | GGAATTCCACAGCCGCAGAAGGGC | 37 | EcoRI | CCCAAGCTTCTCTAGGTCCCGGGGC | 38 | HindIII |
| | | | CGGAAGCCCCGGGACCTAGAGAAG | | | TTCCGGCCCTTCTGCGGCTGTGGAA | | |
| | | | CTTGGG | | | TTCC | | |
| 20 | Elk-1-c-Myc | 145-157 | GGAATTCCATTGGCAGCAAGCAGCG | 39 | EcoRI | CCCAAGCTTGCCCGATGCCATGTAC | 40 | HindIII |
| | | (Mutant) | CAAACGAGTACATGGCATCGGGCA | | | TCGTTTGCGCTGCTTGCTGCCAATG | | |
| | | | AGCTTGGG | | | GAATTCC | | |
| 21 | Elk-1-c-Myc | 145-428 | GGAATTCCATTGGCAGCAAGCAGCG | 41 | EcoRI | CCCAAGCTTTGGCTTCTGGGGCCCT | 42 | HindIII |
| | | (Mutant) | CAAACGAGTACATGGCATCGGGCCT | | | GG | | |
| | | | CTAT | | | | | |
| 22 | FLAG-Elk-1 | 1-428 | GGGGTACCCCAAATGGACCCATCTG | 43 | KpnI | CGGGATCCCGTCATGGCTTCTGGGG | 44 | BamHI |
| | | | TGACG | | | CC | | |
| 23 | PKCα | 1-672 | GGAATTCCAATGGCTGACGTTTTCC | 45 | EcoRI | CCCAAGCTTTACTGCACTCTGTAAGA | 46 | HindIII |
| | | | CGGGC | | | TGGG | | |

*The DNA sequencing of all genes or fragments are completed by Life Technologies (Taipei, Taiwan) and the images of the data are archived by Chromas (Technelysium software for DNA sequence, South Brisbane QLD, Australia) and the gene sequence is identified by NCBI (http://blast.ncbi.nlm.nih.gov/Blast.cgi) as followings.
A.A., amino acids.

Cell Culture and Growth Conditions

Cancer cells from various human organs, namely, hepatocellular carcinoma (HCC) HA22T (BCRC no. 60168), Hep3B (BCRC no. 60434), and HepG2 (BCRC no. RM60025) cells from the liver; breast cancer Hs578T (BCRC no. 60120), MDA-MB-231 (BCRC no. 60425), and MCF-7 (BCRC no. 60436) cells from the breast; and HEK-293 (BCRC no. 60019) cells from embryonic kidney, were purchased from the Bioresources Collection and Research Center, Food Industry Research and Development Institute (Hsinchu, Taiwan). MDA-MB-468 (ATCC no. HTB-132) cells from breast, as well as SK-Hep-1 (ATCC no. HTB-52) and Huh-7 (ATCC no. JCRB-0403) cells from the liver were obtained directly from the ATCC (Manassas, Va., USA). All cultured in media specific to each cell line and supplemented with 10% fetal bovine serum, 100 units/ml penicillin G, and 100 μg/ml streptomycin (Gibico, grand Island, N.Y., USA) in a humidified atmosphere containing 5% CO₂ at 37° C.

Immunohistochemical Analyses

The slide of human cancer tissue recognized by anti-PKC α antibody (BD Biosciences), anti-Elk-1 antibody (Santa Cruz) or anti-MZF-1 antibody (Santa Cruz). PKC α/Elk-1/MZF-1 expression was scored by staining as follows: 1+, weak; 2+, moderate; and 3+, strong.

Chromatin Immunoprecipitation Assay (ChIP)

Cells were harvested and cross-linked with 1% formaldehyde for 10 minutes and the reaction was terminated by the addition of glycine. Cells were washed three times with ice-cold PBS, resuspended in lysis buffer (0.1% SDS, 1% sodium deoxycholate, 150 mM NaCl, 10 mM NaPO₄ (pH 7.2), 2 mM EDTA, 0.2 mM NaVO₃, and 1% NP-40) with complete protease inhibitors (Roche Diagnostics, Mannheim, Germany) and sonicated to shear chromatin using a Cole Parmer Ultrasonic processor (Cole Parmer, Ill., USA). The samples were pre-cleared with protein A agarose (Sigma-Aldrich) for 30 min at 4° C. and incubated with anti-MZF-1, or anti-Elk-1 antibodies (Santa Cruz) overnight at 4° C. The region between −760 and −550 of the PKCα promoter was amplified from the immunoprecipitated chromatin using the primers: sense, 5′-GGTACAGGCAGCTAAAACAC-3′ (SEQ ID NO: 47), and antisense, 5′-GTCTTCCTTCTCCCACTCC-3′ (SEQ ID NO: 48). After PCR, the 210 bp product was resolved and visualized on a 2% agarose gel.

For re-ChIP, the precipitated complexes eluted from the primary immunoprecipitates were pooled from three or four reactions and incubated with 30 μl ChIP elution buffer (50 mM NaHCO₃, 1% SDS). The samples were mixed for 30 minutes at room temperature, centrifuged and the supernatants were collected. The complexes were eluted twice and both eluates were combined. The pooled eluates were diluted 1:10 in a buffer (1% Triton X-100, 5 mM EDTA, 150 mM NaCl, and 25 mM Tris, pH 8) containing a protease inhibitor mixture (Roche Diagnostics). Further supernatant Re-ChIP assays and result analyses were performed as previously described for primary ChIP immunoprecipitation.

Electrophoretic Mobility Shift Assay (EMSA)

EMSA analysis was performed using a LightShift™ chemiluminescent EMSA kit (Pierce). 15 μg of nuclear extract was used for each EMSA analysis. Biotin-labeled double-stranded wild-type MZF-1/Elk-1 oligonucleotides (sense 5′-CCTGAGGATGGGGAAGGGGCTTCCTGCTGCGGTG-3′ as SEQ ID NO: 49, and anti-sense 5′-CACCGCAGCAGGAAGCCCCTTCCCCATCCTCAGG-3′ as SEQ ID NO: 50) containing the MZF-1 and Elk-1 binding sites in the human PKCα promoter; mutant MZF-1/Elk-1 oligonucleotides (sense 5′-CCTGCGTATTTTTAAGGGGCTTCCTGCTGCGGTG-3′ as SEQ ID NO: 51, and anti-sense 5′-CACCGCAGCAGGAAGCCCCTTAAAAATACGCAGG-3′ as SEQ ID NO: 52); MZF-1/mutant Elk-1 oligonucleotides (sense 5′-CCTGCGGATGGGGAAGGGGATTAATGATGAGGTG-3′ as SEQ ID NO: 53, and anti-sense 5′-CACCTCATCATTAATCCCCTTCCCCATCCGCAGG-3′ as SEQ ID NO: 54); and mutant MZF-1/mutant Elk-1 oligonucleotides (sense 5′-CCTGCGTATTTTTAAGGGGATTAATGATGAGGTG-3′ as SEQ ID NO: 55, and anti-sense 5′-CACCTCATCATTAATCCCCTTAAAAATACGCAGG-3′ as SEQ ID NO: 56). The competition study was conducted with a 20- or 100-fold excess of unlabeled wild-type, mutant MZF-1/Elk-1, MZF-1/mutant Elk-1, or mut MZF-1/mut Elk-1 oligonucleotide probes. After the reaction was complete the DNA-protein complexes were electrophoresed and subjected to a 6% native polyacrylamide gel in 0.5× Tris borate/EDTA buffer at 100 V for 3 h and then transferred onto a positively-charged nylon membrane (Hybond™-N⁺) in 0.5× Tris borate/EDTA buffer at 100 V for 1 h. The membrane was immediately cross-linked at 120 mJ/cm² using an UV transilluminator and then analyzed via chemiluminescence according to the manufacturer's instructions.

Plasmid Transfection

Cells were cultured in 60 mm dishes containing minimum essential Dulbecco's Modified Eagle medium (DMEM) supplemented with 10% fetal calf serum (FCS) at 37° C. for 18 hours before rinsing with serum-free DMEM. Then the sample was transferred to 1 ml serum-free MEM containing 15 μg Lipofectamine 2000 transfection reagent (Invitrogen) and various doses of the indicated plasmid. After incubating for a minimum of 6 hours, 1 ml DMEM supplemented with 20% FCS was added to the medium. After incubating for another 18 hours the medium was replaced with fresh FCS-DMEM, followed by incubation for at least 48 hours before the cells were lysed for subsequent assays.

Stable Clone Establishment

Stable clones were established by seeding low-passage cells at a density of 3×10⁵ cells in 60-mm tissue culture dishes and transfecting the cells with 5 μg MZF-1₆₀₋₇₂ plasmid using Lipofectamine 2000. Stable clones were selected by growing the cells at 1:10 to 1:15 (vol/vol) in DMEM supplemented with geneticin (G418; 600 μg/ml) at 37° C. for five weeks. Individual clones were then transferred to 96-well plates and grown until confluence. After being transferred to flasks the cells were cultured until confluence, harvested and frozen in liquid nitrogen for further experiments.

TAT-Fused Peptide

The TAT-fused peptides were designed such that the TAT moiety corresponds to amino acid residues 48-57 of the HIV TAT protein, the MZF-1₆₀₋₇₂ moiety corresponds to residue 60-72 of the human MZF-1 protein and the Elk-1₁₄₅₋₁₅₇ moiety corresponds to residues 145-157 of the human Elk-1 protein. The TAT and MZF-1/Elk-1 moieties were linked by three glycine linker residues. The TAT-fused peptide MZF-1 normal fragment (60-72; YGRKKRRQRRRGGGDEDTPDQESRLDS as SEQ ID NO: 57), MZF-1 mutant fragment (60-72; YGRKKRRQRRRGGGAEATPAQESRLAS as SEQ ID NO: 58), Elk-1 normal fragment (145-157; YGRKKRRQRRRGGGLARSSRNEYMRSG as SEQ ID NO: 59), and Elk-1 mutant fragment (145-157; YGRKKRRQRRRGGGLAASSANEYMASG as SEQ ID NO: 60) were synthesized by MDBio, Inc. (Taipei, Taiwan). For transduction of the TAT fusion proteins cells were cultured to 50-60% confluence. The culture medium was removed and replaced with fresh, serum-free medium, followed by the addition of the TAT fusion proteins at the indicated concentrations. Three days post-treatment the cells were used for migration assays and western blotting.

In Vitro Tumorgenesis Assay

Female or male 4- to 6-week-old BALB/c nude mice were purchased from the National Health Research Institute (Taipei, Taiwan) and housed in a dedicated nude mouse facility with microisolator caging. The cancer cells were detached from culture dishes by trypsinization 48 hours later and then washed three times in serum-free DMEM. Approximately 1×10⁷ cells in 100 μl volume were subcutaneously injected into the right posterior flank of the mice using a 1 ml syringe with a 24-gauge needle. Five mice were used in each group, and the experiment was repeated twice. The tumor volume was calculated using the formula 0.5236×L1 (L2)², where L1 is the long diameter and L2 is the short diameter. The inhibition of tumor growth was calculated using the following formula: (tumor volume in control group−tumor volume in test group)/(tumor volume in control group)×100%. After 2 or 3 months the mice were sacrificed to remove the tumors and the tumor mass was measured and subjected to histopathological examination.

Cell Proliferation Assay

Cell proliferation was analyzed using the yellow tetrazolium (MTT) assay. Cells were seeded in 24-well plates at 1×10⁴ cells/well and cultured in DMEM containing 10% FCS at 37° C. overnight. Cells were treated with or without various plasmids and incubated for 24 or 48 h. After incubation, the medium was replaced with fresh medium, and the cells were incubated with 1 mg/ml MTT for 3 h before being dissolved in 1 ml of DMSO for 30 min. The optical density at 570 nm was measured using a spectrophotometer.

Cell Migration Assay

The migration assay was performed using a 48-well Boyden chamber (Neuro Probe) plated with 8-μm pore size polycarbonate membrane filters (Neuro Probe). The lower compartment was filled with DMEM containing 20% FCS. Cells were placed in the upper part of the Boyden chamber and incubated for 12 hours. After incubation the cells were fixed with methanol and stained with 0.05% Giemsa for 1 hour. The cells on the upper surface of the filter were removed with a cotton swab. The filters were then rinsed in distilled water until no additional stain leaching was observed. The cells were air-dried for 20 minutes. The migratory phenotypes were determined by counting the cells that migrated to the lower side of the filter through microscopy at 200× magnification.

Cell Invasion Assay

The invasion assay was performed using a 48-well Boyden chamber with polycarbonate filters. The upper side was pre-coated with 10 μg/ml Matrigel (BD Biosciences). Cells were placed in the upper part of the Boyden chamber and incubated at 37° C. for 24 hours. The experimental procedures were identical to the migration assay procedures.

Antisense Knockout Assay

The antisense knockout assay was performed with the following antisense and sense (as a control) sequences: Elk-1 (antisense 5′-CAGCGTCACAGATGGGTCCAT-3′ as SEQ ID NO:61, and sense 5′-ATGGACCCATCTGTGACGCTG-3′ as SEQ ID NO:62), and MZF-1 (antisense 5′-TACACAAGGGGACCATTCATTC-3′ as SEQ ID NO:63, and sense 5′-GAATGAATGGTCCCCTTGTGTA-3′ as SEQ ID NO:64).

### Example 1

### Expression of PKCα Correlates with MZF-1/Elk-1

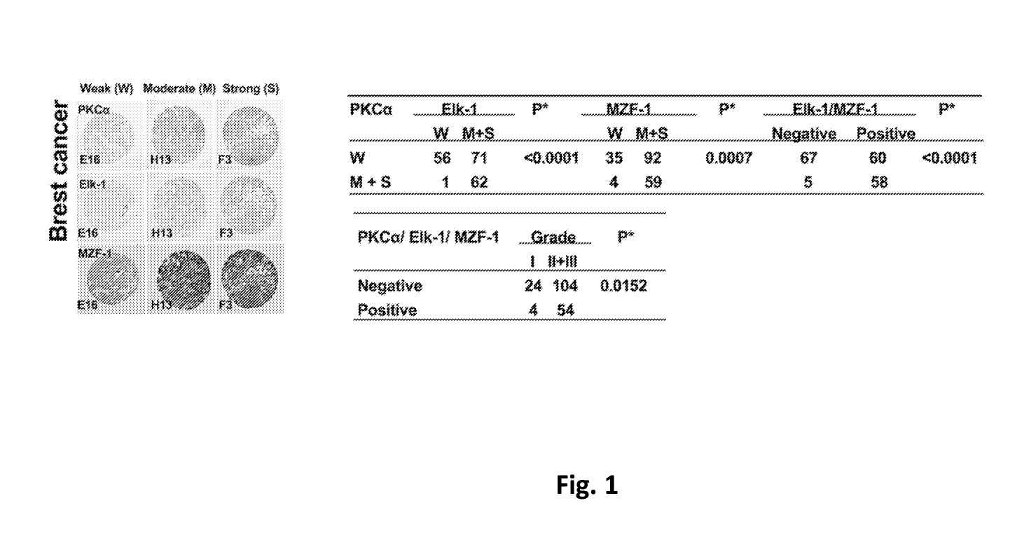

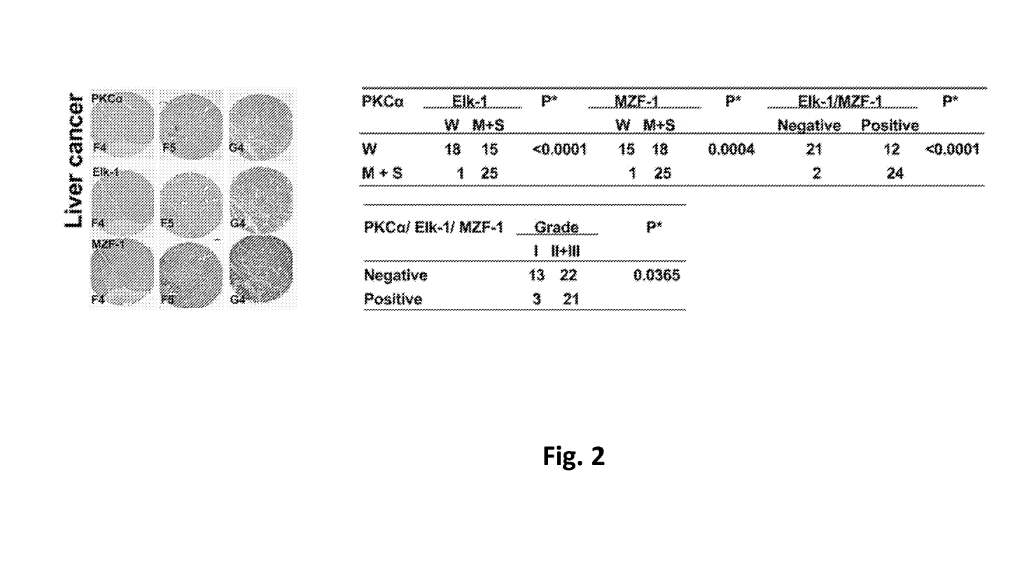

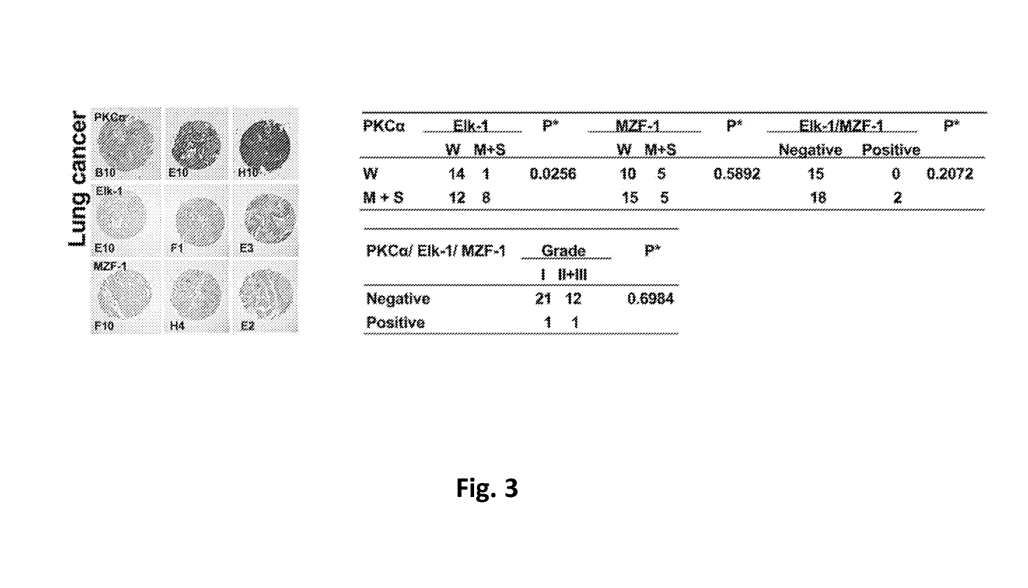

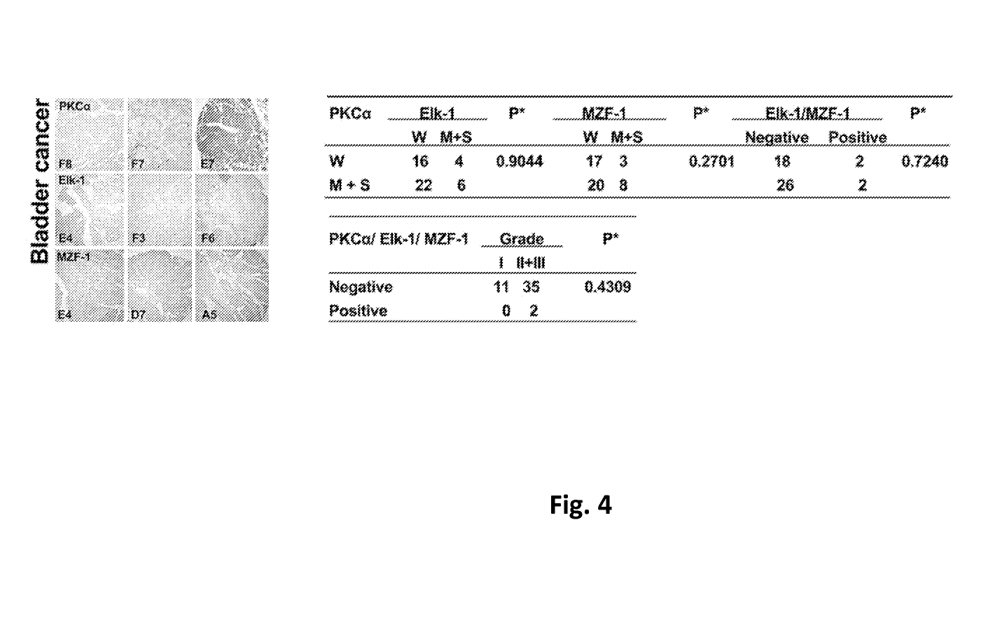

To determine whether the clinical relevance of the correlation between PKCα and Elk-1 and/or MZF-1 exists in cancers with tissue specificity, the expression of PKCα, Elk-1 and MZF-1 in tissue arrays of human breast, liver, lung, and bladder cancers were analyzed by immunohistochemical (IHC) staining. A positive correlation was observed between moderate-to-strong PKCα and either Elk-1 and/or MZF-1 staining in breast (FIG. 1) and liver (FIG. 2) but not lung (FIG. 3) or bladder (FIG. 4) cancers. Moreover, moderate-to-strong staining of PKCα/Elk-1/MZF-1 was most common in grade 2 and grade 3 breast and liver cancers. In cell lines model which knockdown assay by siRNA Elk-1 decreased PKCα protein expression in TNBC MDA-MB-231 (MB-231) and liver SK-Hep-1 cancer cell, but not in lung A549 and bladder 5637 cancer cells, suggesting that PKCα along with Elk-1/MZF-1 function as important mediators of tumor progression in respective cancers (including breast and liver cancers) and supporting the PKCα gene regulation in different cell types may be due to cell-specific factors.

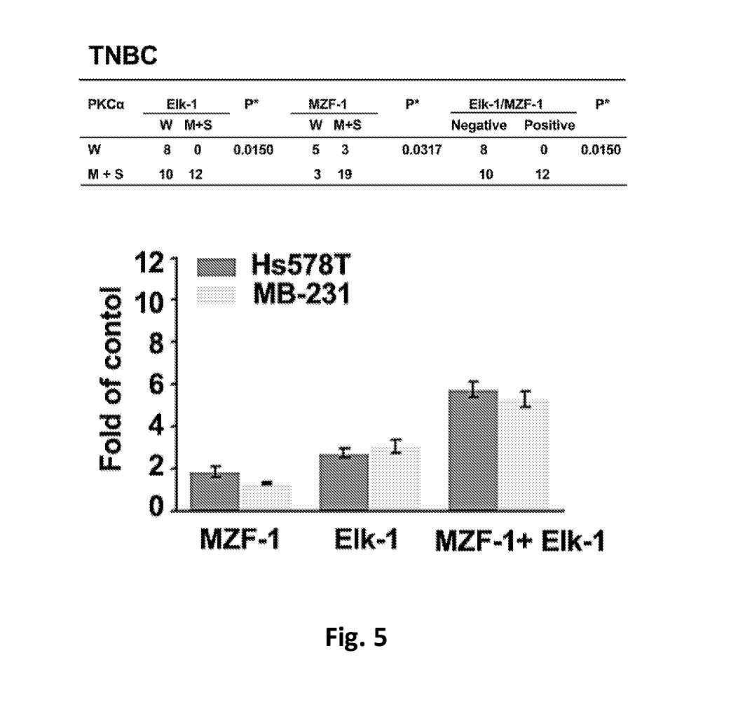

The same proteins were detected in tissue array of TNBC in which the correlations between moderate-to-strong PKCα and either Elk-1 and/or MZF-1 staining were also observed (FIG. 5). To demonstrate PKCα transcriptional activity increased by Elk-1/MZF-1 significantly in TNBC we evaluated its promoter containing a putative binding site for MZF-1/Elk-1 by a luciferase reporter assay in TNBC MDA-MB-231/Hs578T cells as previously described. As shown in FIG. 5, the fold increases in the transcriptional activities by Elk-1 or/and MZF-1 were also there, further supporting the role of Elk-1/MZF-1 in regulating PKCα expression in TNBC.

Thus Elk-1/MZF-1 regulates PKCα expression in liver cancer, breast cancer and even TNBC cells.

### Example 2

### MZF-1/Elk-1 Complex Binds to the Promoter Region of PRKCA

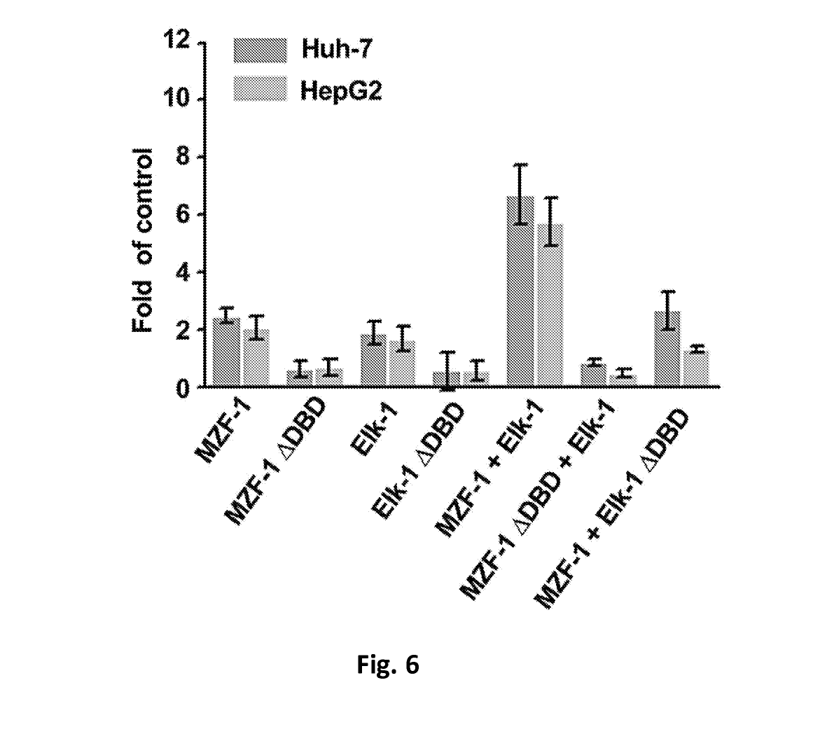

To further determine if MZF-1/Elk-1 bind directly to the PRKCA promoter to regulate its transcriptional activity, we constructed deletion mutants of Elk-1 (Elk-1 ΔDBD; Elk-1₈₇₋₄₂₈) and MZF-1 (MZF-1ΔDBD; MZF-1₁₋₇₂) lacking the DNA-binding domain(s). Co-transfection of full-length MZF-1 or Elk-1 in two HCC cell lines (Huh-7 and HepG2) increased PKCα transcriptional activity as indicated by luciferase reporter activities but not the corresponding deletion mutant lacking the DNA-binding domain (FIG. 6). Cells expressing both full-length MZF-1 and Elk-1 but not expressing Elk-1ΔDBD (Elk-1₈₇₋₄₂₈) or MZF-1ΔDBD (MZF-1₁₋₇₂), had significantly higher PKCα transcriptional activity compared with expression of each alone. These results shown MZF-1/Elk-1 bind directly to the PRKCA promoter to regulate transcriptional activity of PKCα.

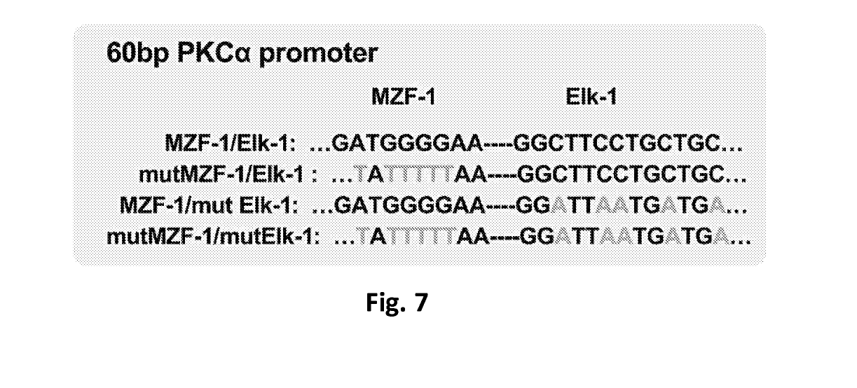

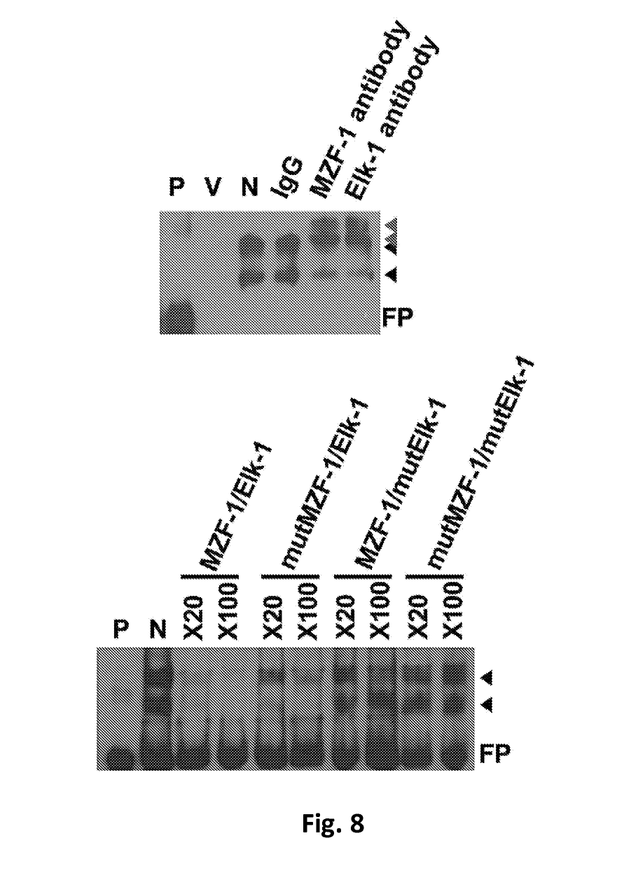

Mutate the PRKCA promoter region by replacing all guanine bases with thymines and all cytosines with alanines (FIG. 7), and conducted electrophoretic mobility shift assay (EMSA) by incubating nuclear extract of cancer cells with biotinylated wild-type double-stranded oligonucleotide probes containing both the Elk-1 and MZF-1 binding sequences of the PRKCA promoter. As shown in FIG. 8, we identified two slow migrating bands and incubation with antibody against either MZF-1 or Elk-1 resulted in two supershifted bands in TNBC (MDA-MB-231) cell model (P: probe only; V: nuclear extract only; N: nuclear extract plus probe; FP: free probe.).

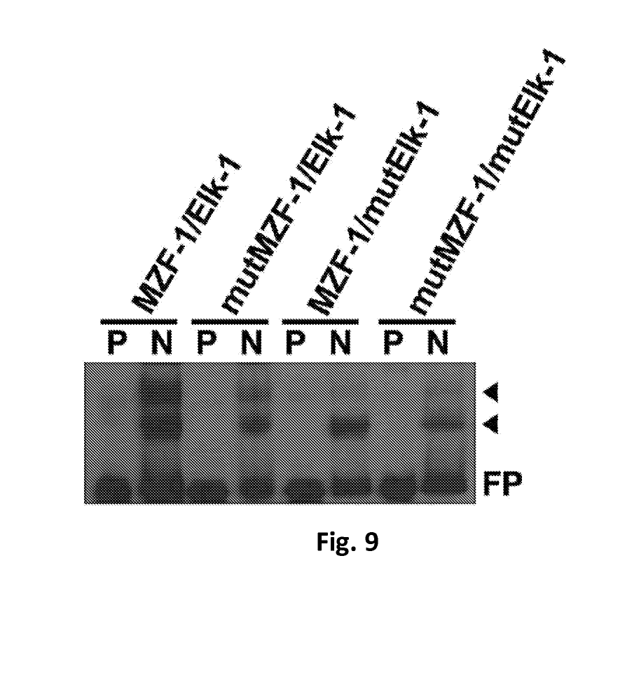

In contrast, binding was reduced when we incubated the nuclear extract with mutant probes with alterations in the Elk-1 and/or MZF-1 binding sites (FIG. 9: P: probe only; N: nuclear extract plus probe; FP: free probe). Moreover, the addition of a 20-fold and 100-fold excess of unlabeled wild-type probes more substantially decreased binding than with unlabeled mutant probes (mut MZF-1, mut Elk-1, or mut MZF-1/Elk-1). Together, these findings provide evidence to demonstrate that MZF-1/Elk-1 bind to the PRKCA promoter and regulate its transcriptional activity.

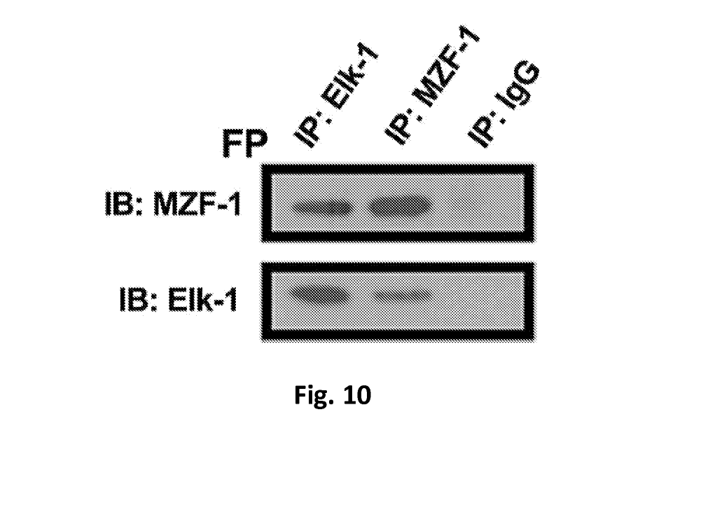

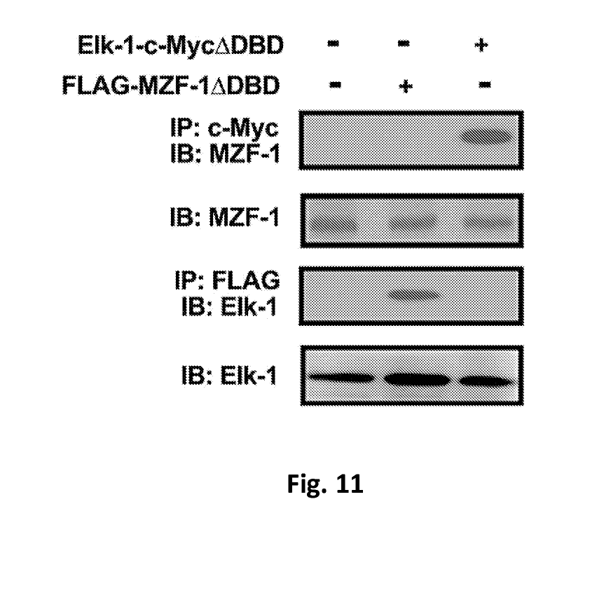

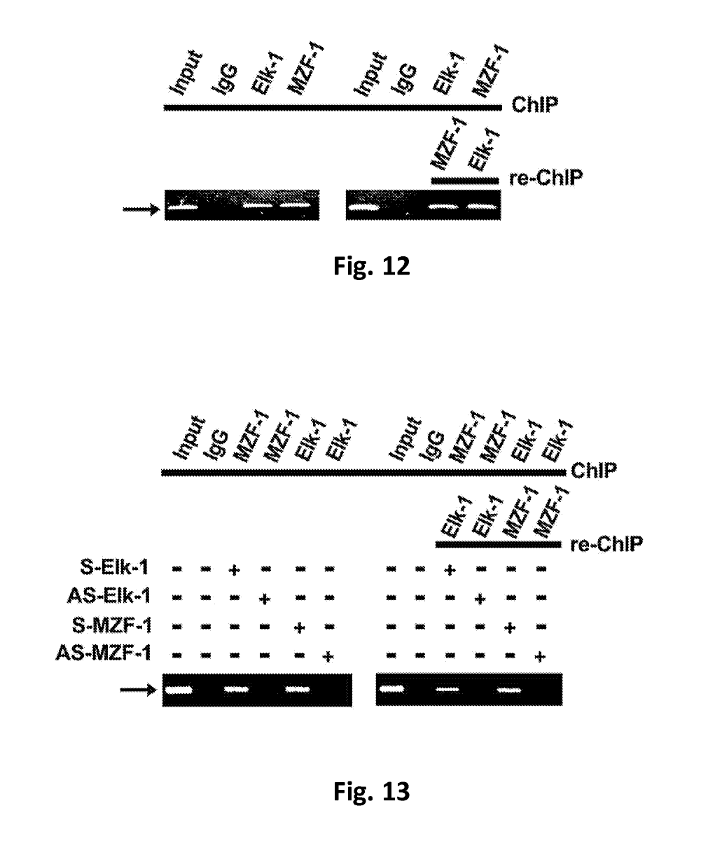

Because the Elk-1/MZF-1 DNA-binding sites are proximal on the PRKCA promoter, we hypothesized that MZF-1/Elk-1 form a heterodimeric complex. To this end, we conducted co-immunoprecipitation and identified MZF-1 in the complex by the Elk-1 antibody and vice versa (FIG. 10). In addition, we transfected cells with truncated Elk-1 (Elk-1-c-Myc-ΔDBD deletion mutant lacking the N-terminal region), the MZF-1 protein was observed in the complex immunoprecipitated by a c-Myc antibody (FIG. 11). Similarly, when the cells were transfected with Flag-MZF-1ΔDBD vector the Elk-1 protein was observed in the complex immunoprecipitated by a FLAG antibody. The presence of MZF-1/Elk-1 in all cells in this experiment indicates that Elk-1 binds to the N-terminal region of MZF-1 and that MZF-1 binds to the C-terminal region of Elk-1, forming a heterodimeric complex. To determine if the Elk-1/MZF-1 heterodimer forms before binding to PRKCA promoter we carried out a chromatin immunoprecipitation (ChIP) assay. As shown in FIG. 12, the PRKCA promoter fragment was amplified from the immunoprecipitated complex using either Elk-1 or MZF-1 antibody. Results from the re-Chip assay indicated that the MZF-1/Elk-1 form a complex and bind to PRKCA promoter. Knocking down Elk-1 or MZF-1 by transfection with antisense oligonucleotides blocked amplification of the PRKCA promoter fragment pulled down by MZF-1 or Elk-1 antibody, respectively (FIG. 13). These findings suggest that formation of the MZF-1/Elk-1 heterodimer is required for its binding to the promoter region of PKCα.

### Example 3

### The Acidic Domain of MZF-1 Interacts with the Heparin-Binding Domain of Elk-1

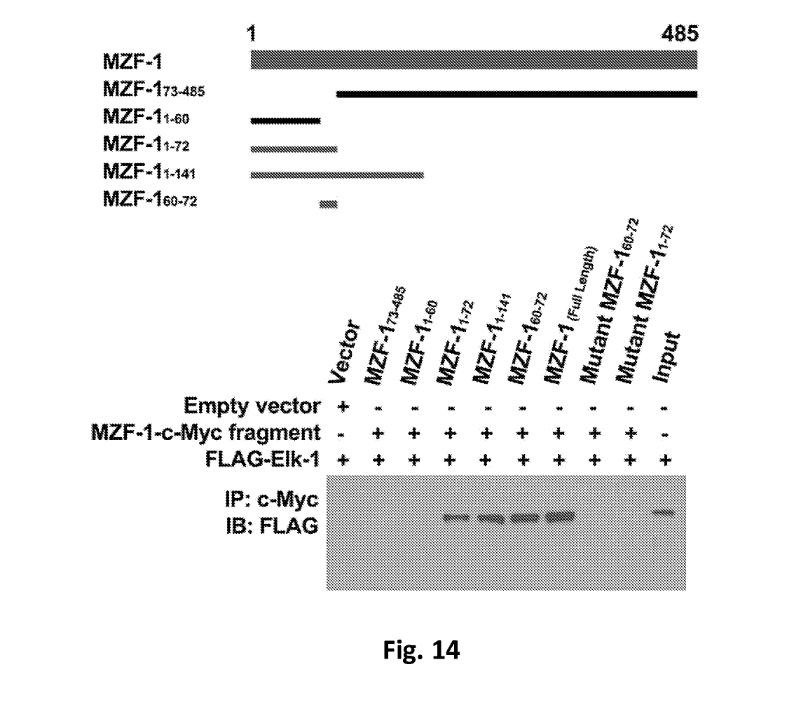

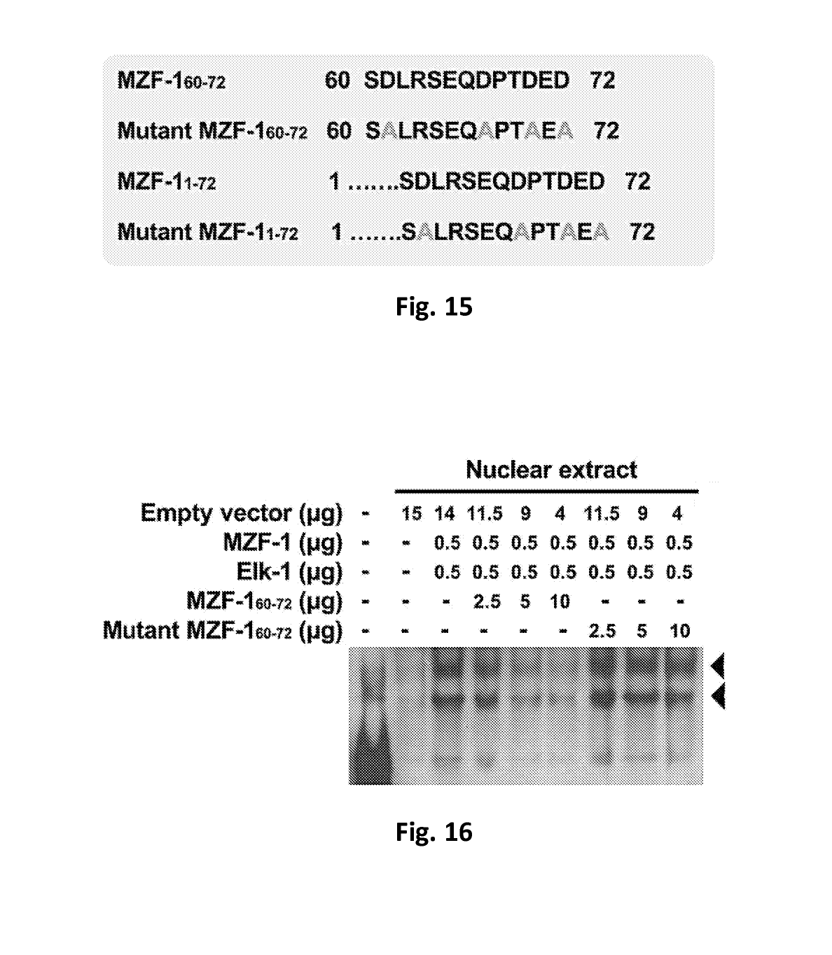

MZF-1 contains an acidic domain (amino acids 60-72) with six aspartates or glutamates upstream of the zinc finger regions. To identify the specific residues through which MZF-1 interacts with Elk-1 we designed various protein fragments containing only the relevant interacting domains for co-immunoprecipitation assays (FIG. 14, top). The full-length MZF-1 and MZF-1₁₋₇₂, MZF-1₁₋₁₄₁, and MZF-1₆₀₋₇₂ fragments (all contain the acidic domain) all bound Elk-1 (FIG. 14, lower panel) but not MZF-1₁₋₆₀ or MZF-1₇₃₋₄₈₅. We also generated mutations within MZF-1₆₀₋₇₂ and MZF-1₁₋₇₂ in which the negatively charged aspartates (D61, D67, D70, and D72) were changed to uncharged alanine and found that their interaction with Elk-1 was substantially decreased (FIG. 15).

We also disrupted the interactions between endogenous Elk-1 and MZF-1 by saturating the protein-protein binding domains with peptides corresponding to the MZF-1₆₀₋₇₂ fragment. Results from EMSA demonstrated that MZF-1₆₀₋₇₂ decreased Elk-1 and MZF-1 DNA-binding activity in a dose-dependent manner (FIG. 16). The mutant form of the fragment did not affect their binding activity. Together these findings further validated that MZF-1 interacts with Elk-1 through its acidic domain.

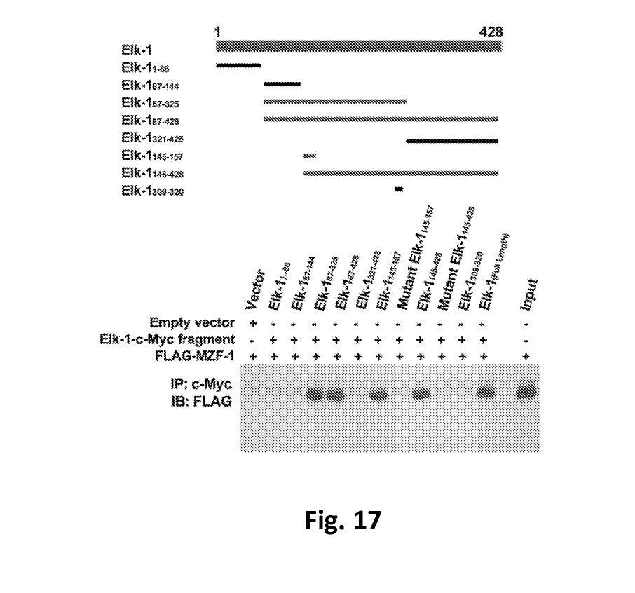

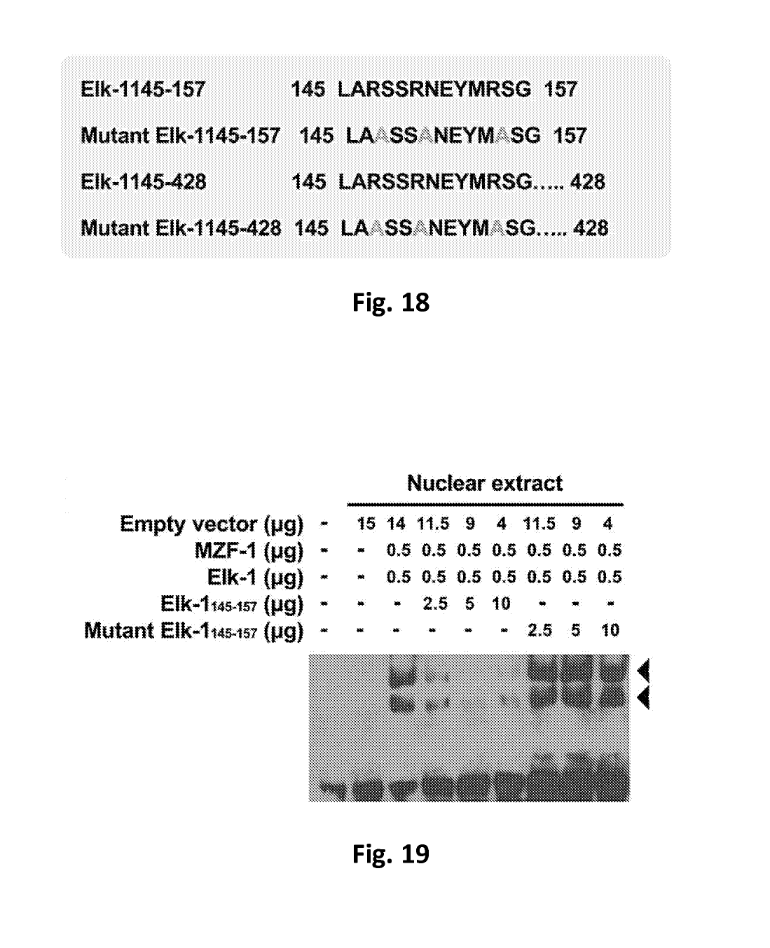

Likewise, we also constructed several Elk-1 fragments to determine the region through which Elk-1 interacts with MZF-1 (FIG. 17, top). The fragments that contained amino acids 145-157 interacted with MZF-1 (FIG. 17, bottom) whereas mutation of the positively charged arginines to alanine (R147, R150, and R155) within this region abolished their interaction (FIG. 18). Moreover, addition of the Elk-1₁₄₅₋₁₅₇ fragment also decreased Elk-1 and MZF-1 DNA-binding activity in a dose-dependent manner (FIG. 19) while the mutant form did not. These findings identify the region spanning amino acids 145-157 as the heparin-binding domain binds to MZF-1.

### Example 4

### Inhibition of MZF-1 and Elk-1 Heterodimer Formation Attenuates Drug Resistance and Malignant Phenotypes by Reducing PKCα Expression

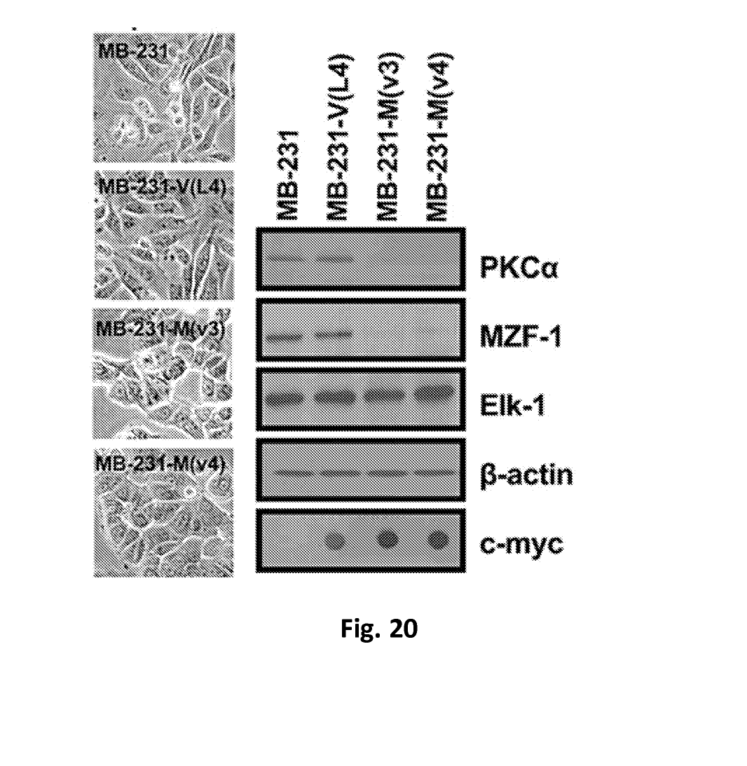

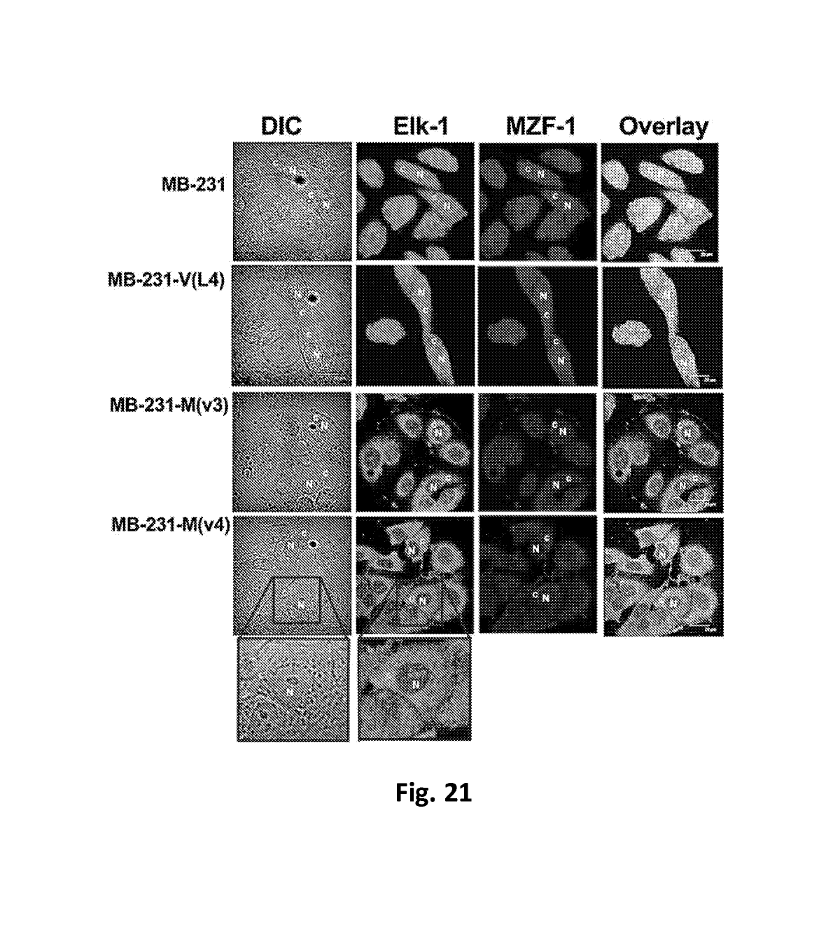

The effect of MZF-1₆₀₋₇₂ on PKCα expression was investigated because it competes with endogenous MZF-1 for Elk-1 binding and decreases endogenous DNA-binding activity. The results showed that TNBC MDA-MB-231 and Hs578T breast cancer cells stably expressing MZF-1₆₀₋₇₂ [MDA-MB-231-M(v3), MDA-MB-231-M(v4), Hs578T-M(s2) and Hs578T-M(s3)] were more rounded compared with the elongated parental and vector control cells (FIG. 20). In addition, PKCα and MZF-1 expressions decreased, whereas Elk-1 levels remained relatively the same, and both were predominantly present in the cytosol of the stable cells but more equally distributed in the nucleus and cytosol of the parental and empty vector cells. Fluorescence imaging of confocal immunofluorescence microscopy analysis confirmed the cytosolic localization of Elk-1/MZF-1 in the stable cells (FIG. 21: “N” indicates the nucleus; “C” indicates the cytosol).

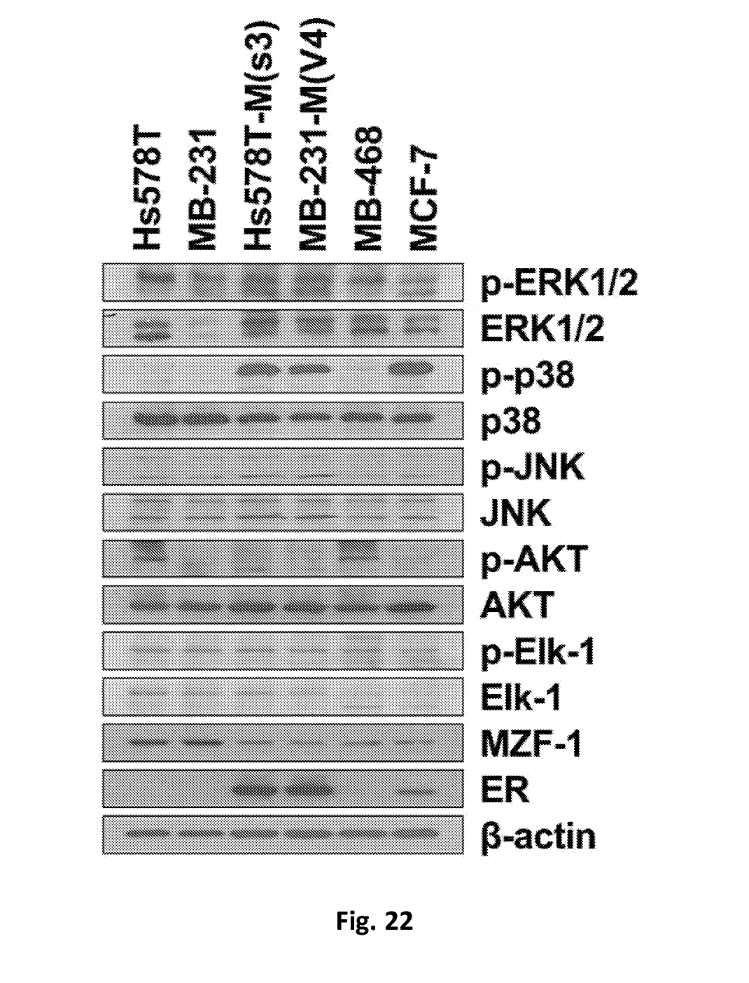

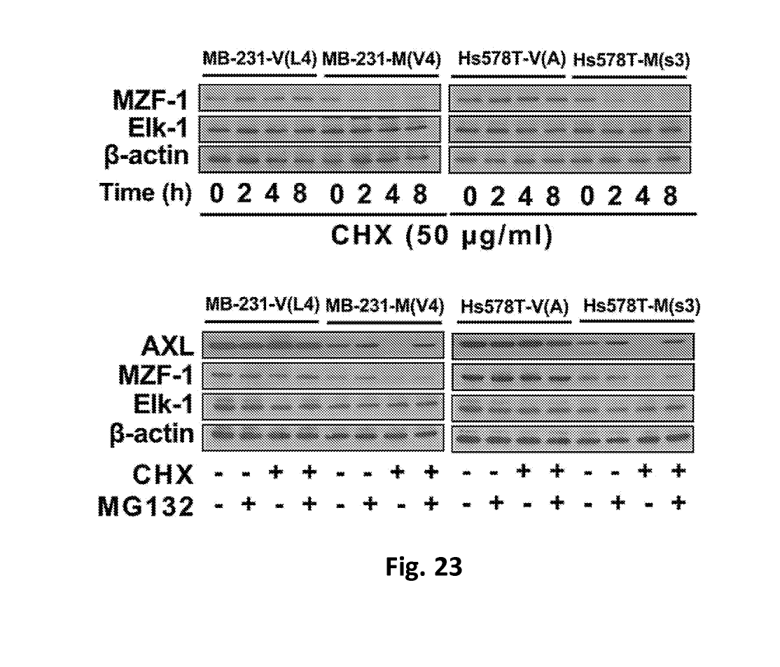

Immunoblotting data showed no change in Elk-1 phosphorylation in stable cells (FIG. 22) but prohibit the nuclear translocation. The decrease in MZF-1 was due to increased protein degradation, which is determined by cycleheximide and proteasome inhibitor (MG132) treatment (FIG. 23). These observations indicate that the interaction between the two endogenous transcription factors promotes MZF-1 protein stability and their entry into the nucleus.

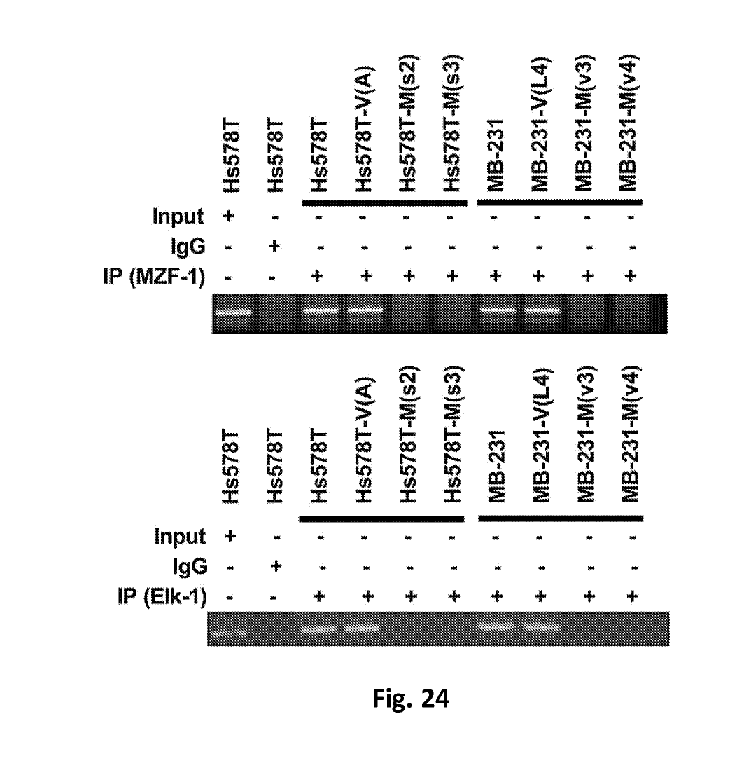

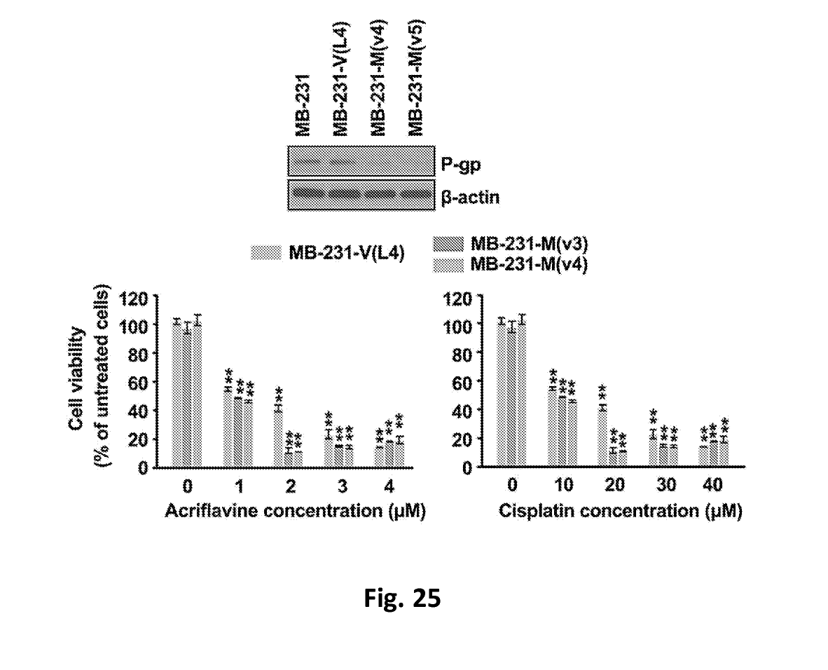

To determine if MZF-1₆₀₋₇₂ blocks endogenous Elk-1 and MZF-1 from binding to the PKCα promoter we carried out ChIP assays. The PKCα promoter fragments amplified from the immunoprecipitated complex using either Elk-1 or MZF-1 antibodies decreased in all MZF-1 MZF-1₆₀₋₇₂-expressing stable MDA-MB-231 cells (FIG. 24). P-glycoprotein 1 (P-gp) is known as multidrug resistance protein 1. The level of P-gp decreased in MZF-1₆₀₋₇₂-expressing stable MDA-MB-231 cells (FIG. 25).

To determine if MZF-1₆₀₋₇₂ peptide-mediated decrease in P-gp expression sensitizes breast cancer cells to chemotherapeutic agents, MZF-1₆₀₋₇₂-expressing MDA-MB-231 stable cells were treated with acriflavine and cisplatin (commonly used to treat breast cancer-insert) and their cell viability measured. The 50% inhibitory concentration (IC₅₀) of acriflavine against these cells was reduced from 2.43 μM to 1.49 μM and cisplatin from 23.74 μM to 16.19 μM. We also observed a decrease in the IC₅₀ in Hs578T cells (2.56 μM to 1.33 μM against acriflavine and 27.97 μM to 21.14 μM against cisplatin). These data indicate that MZF-1₆₀₋₇₂ inhibits endogenous Elk-1 and MZF-1 interaction and subsequent moderate their bindings to the PRKCA promoter, thereby reducing PKCα and P-gp expression and increasing drug sensitivity.

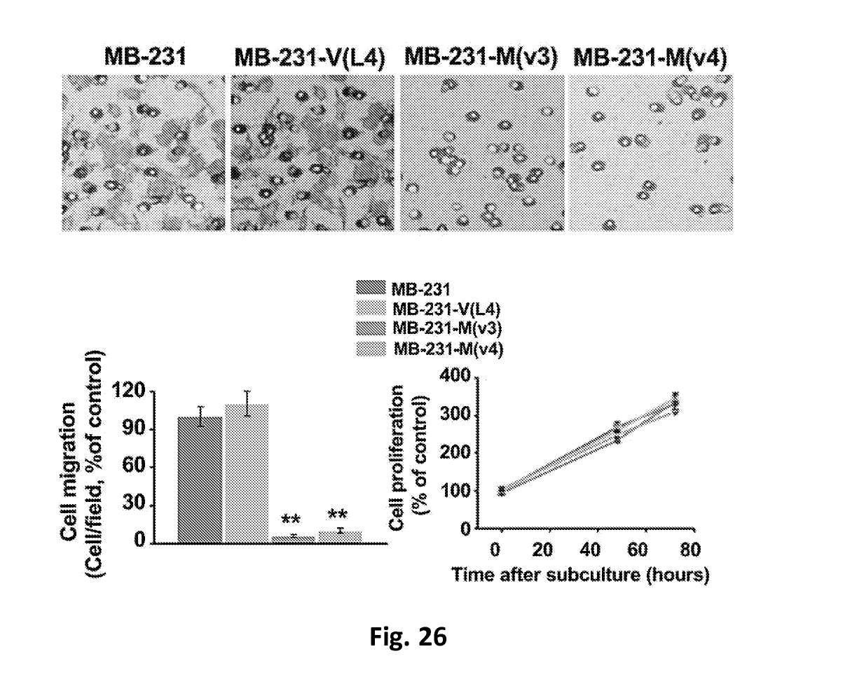

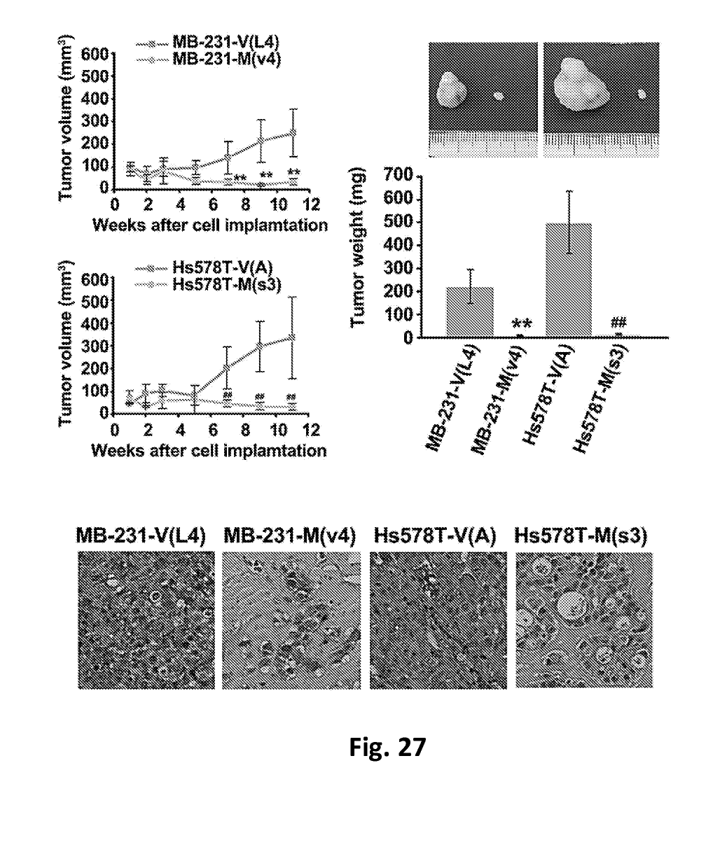

We examined the effects of MZF-1₆₀₋₇₂ on the tumorigenic potential of MDA-MB-231 and Hs578T breast cancer cells. Cell migration was significantly reduced by 80-90% in MZF-1₆₀₋₇₂-expressing stable cells relative to parental and control cells (FIG. 26). However, no changes in cell proliferation were observed. The efficacy of MZF-1₆₀₋₇₂ was then evaluated in a breast cancer xenograft mouse model. Mice injected with MZF-1₆₀₋₇₂-expressing stable MDA-MB-231-M (v4) and Hs578T-M (s3) cells developed tumors more slowly than those injected with vector control cells (FIG. 27, top left). The maximum inhibition of tumor growth was 91.0%±5.2% (n=5) for Hs578T and 90.7%±4.6% (n=5) for MDA-MB-231-M (v4) stable cells. The mean tumor growth inhibition from 7 to 11 weeks was 85.6%±4.1% for Hs578T-M (s3) and 84.4%±6.7% for MDA-MB-231-M (v4) stable cells. Tumor weight from mice injected with MZF-1₆₀₋₇₂-expressing stable cells was also significantly reduced (FIG. 27, right), and the cells isolated from these tumors displayed more interstitial tissue at the beginning of what appeared to be the formation of tubular structures (FIG. 27, bottom). These data shows additional MZF-1₆₀₋₇₂ blocks endogenous Elk-1 and MZF-1 and regulate PRKCA transcriptional activity from binding to the PKCα promoter, and prohibits tumorigenic potential.

### Example 5

### Blocking MZF-1/Elk-1 Heterodimer Formation Decreases EMT Potential

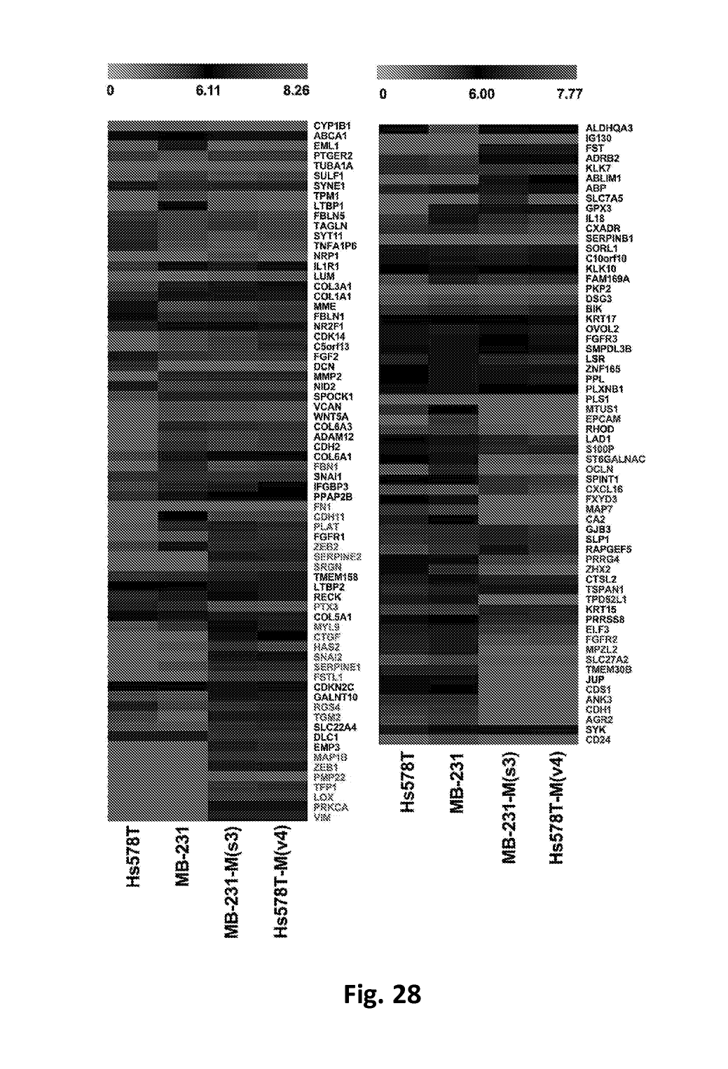

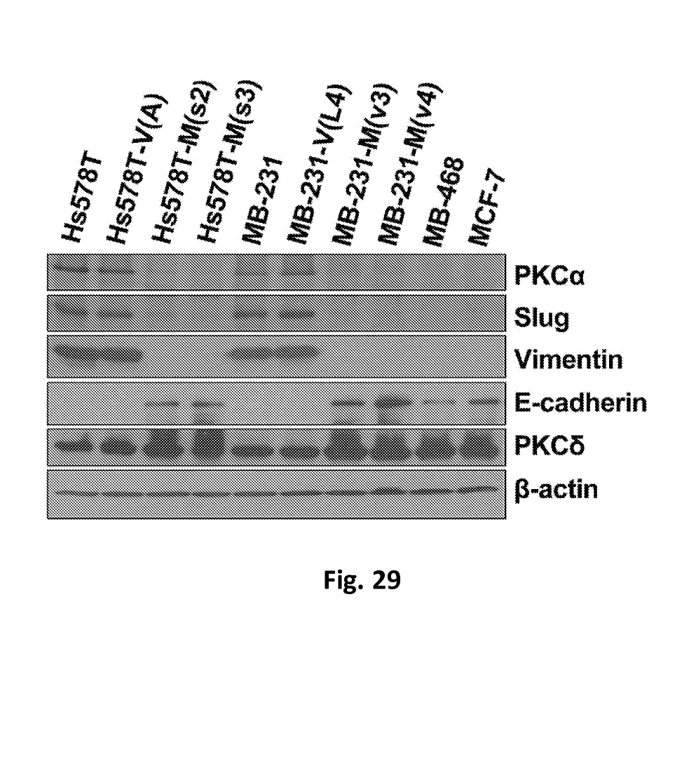

Of the 22,203 genes analyzed in both cell lines, 1209 genes and 1557 genes exhibited a two-fold increase and decrease, respectively, in expression in Hs578T-M(s3) cells (accession number GSE56306). In MDA-MB-231-M(v4) cells, 1272 genes and 1494 genes exhibited a two-fold increase and decrease in expression. Among these, 821 and 931 genes were upregulated or downregulated, respectively, in both cell lines. The affected genes have diverse biological functions, including 24 EMT-core-upregulated genes (CDH11, CTGF, EMP3, FBN1, FN1, FSTL1, HAS2, LOX, MAP1B, MYL9, PLAT, PMP22, PRKCA, PTX3, RGS4, SERPINE1, SERPINE2, SNAI2, SRGN, TFPI, TGM2, VIM, ZEB1, and ZEB2) that were decreased (FIG. 28, left, red) and 24 EMT-core-downregulated genes (AGR2, ANK3, CA2, CD24, CDH1, CDS1, CXCL16, ELF3, EPCAM, FGFR2, FXYD3, JUP, MAP7, MPZL2, MTUS1, OCLN, PRRG4, S100P, SLC27A2, ST6GALNAC2, TMEM30B, TPD52L1, TSPAN1, and ZHX2) that were increased (FIG. 28, right, green). The protein levels of PKCα, Slug (SNAI2) and Vimentin (VIM) were markedly decreased whereas E-cadherin (CDH1) was substantially increased and PKCδ remained unchanged (FIG. 29).

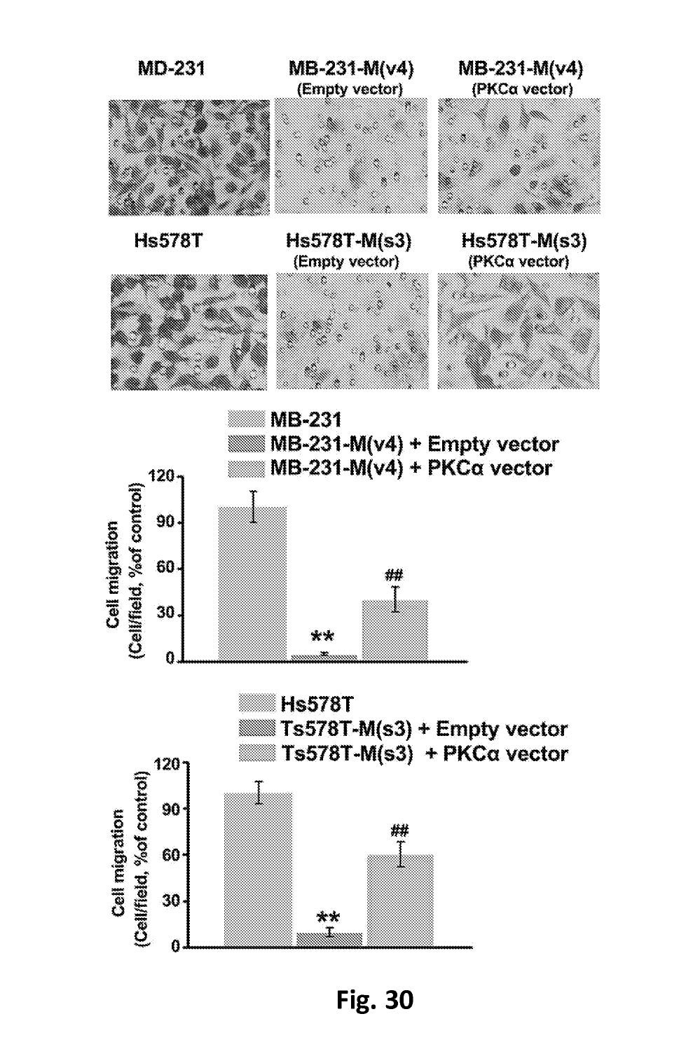

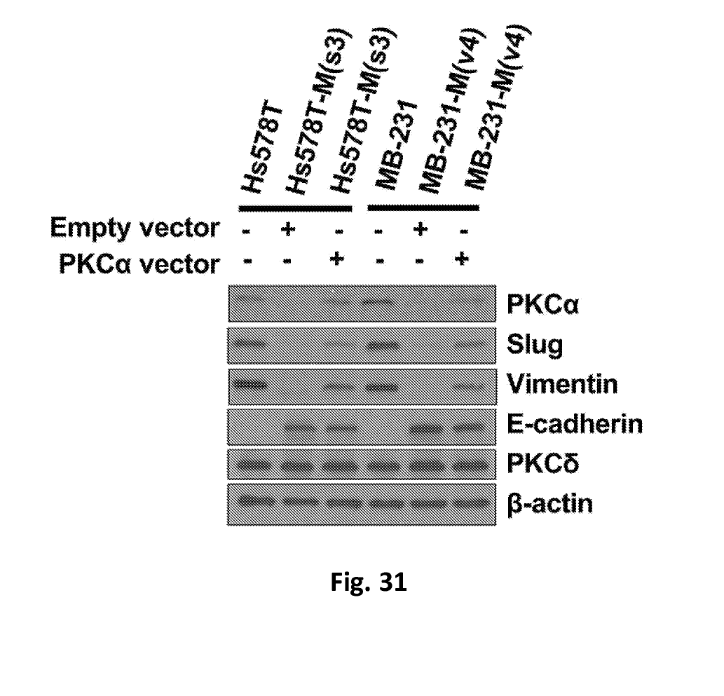

To validate the function of PKCα in EMT, MDA-MB-231-M(V4) and Hs578T-M(s3) cells were transfected with full-length PKCα. Expression of PKCα significantly increased cell migration in MDA-MB-231-M(V4) and Hs578T-M(s3) cells from 5% to 41% and from 9% to 62%, respectively, compared with the untransfected cells (FIG. 30). In addition, the expression levels of the EMT-related genes (Slug, Vimentin and E-cadherin) were also enhanced (FIG. 31).

### Example 6

### TAT-Fused Peptides Inhibit MZF-1/Elk-1 Heterodimer Formation

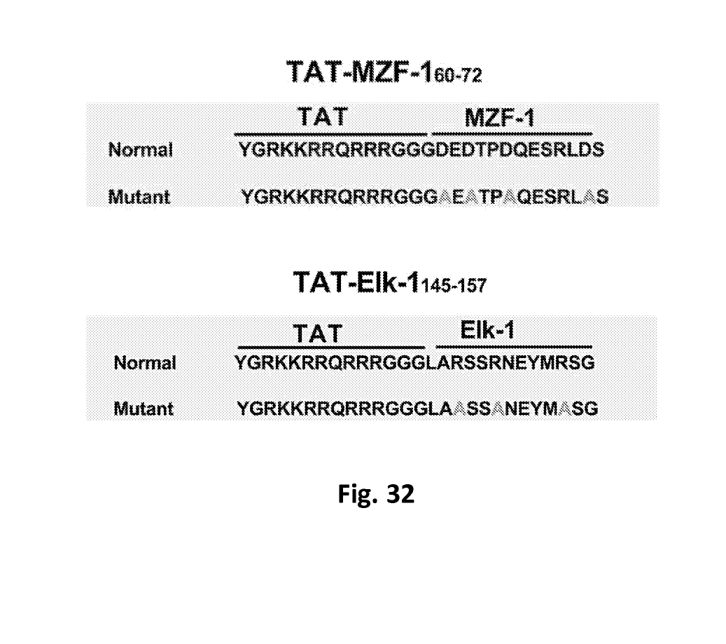

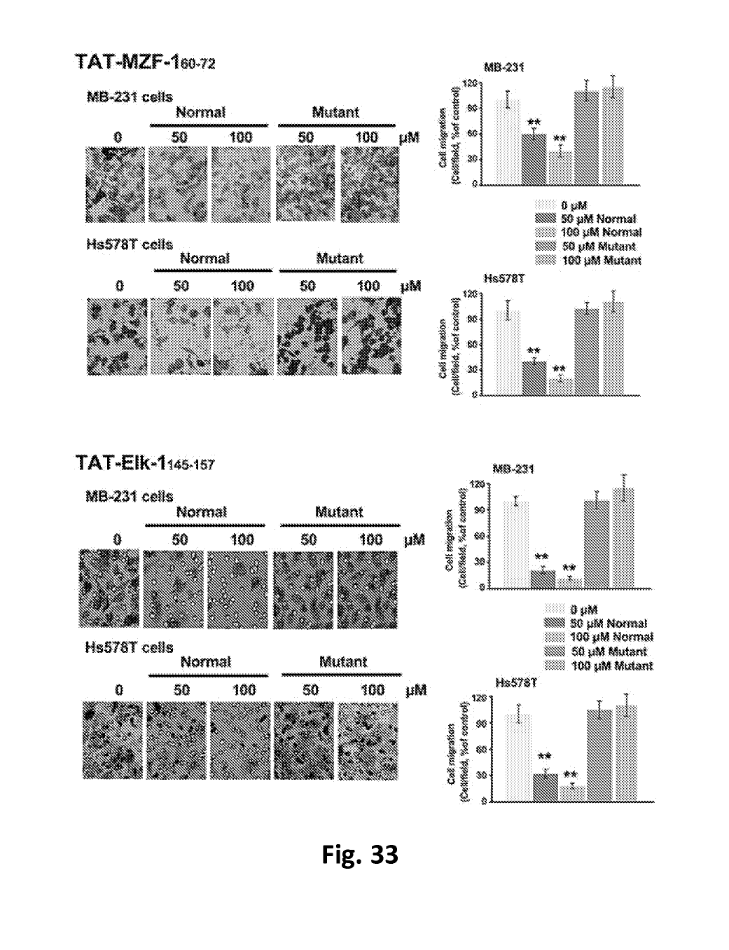

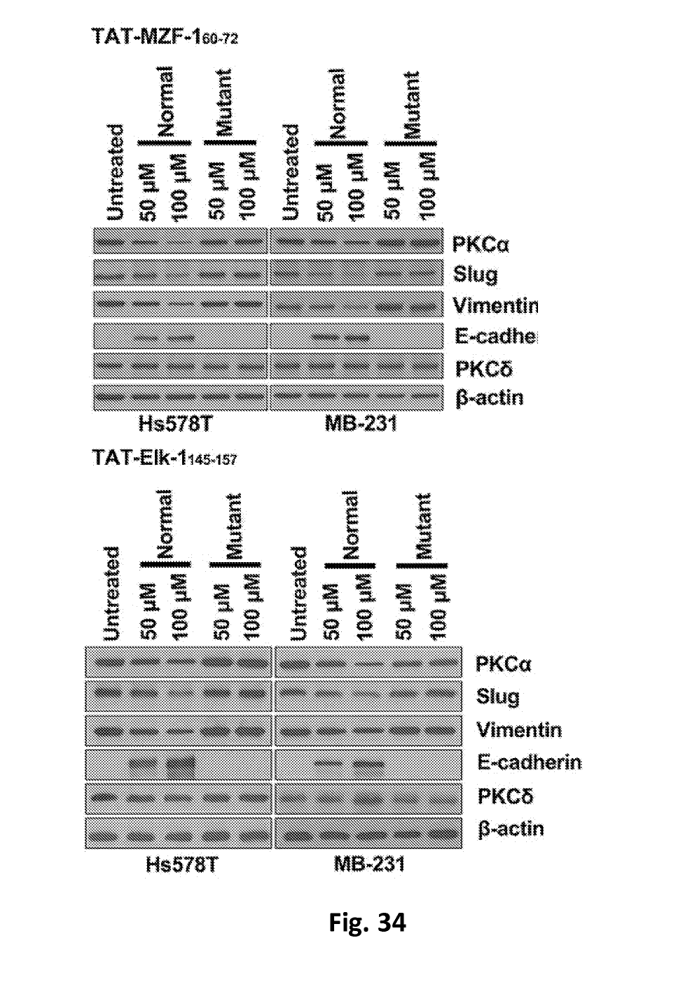

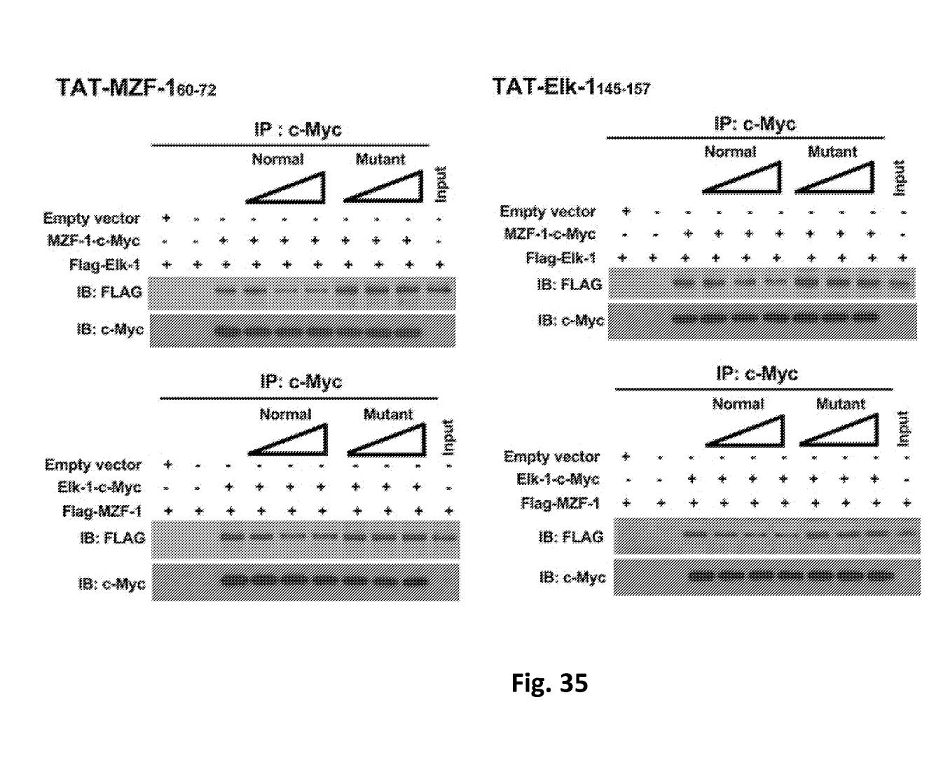

Hs578T and MDA-MB-231 cells treated with either TAT-MZF-1₆₀₋₇₂ or TAT-Elk-1₁₄₅₋₁₅₇ peptide (FIG. 32) had reduced cell migration (FIG. 33). In addition, the levels of EMT-related (PKCα, Slug and Vimentin) and MET-related (E-cadherin) proteins were noticeably increased and decreased, respectively (FIG. 34). However, no changes were observed in cells treated with a TAT-fused mutant peptide (as described in FIG. 3 but fused with TAT). These findings are consistent with prior results (FIG. 16). Moreover, results from co-immunoprecipitation assay indicated that both MZF-1₆₀₋₇₂ and Elk-1₁₄₅₋₁₅₇ but not mutant TAT-fused peptides reduced the binding of MZF-1 to Elk-1 and vice versa in a dose-dependent manner (FIG. 35). Together, these results further demonstrate that the interaction between MZF-1 and Elk-1 plays an important role in the tumor progression and blocking this interaction has therapeutic potential.

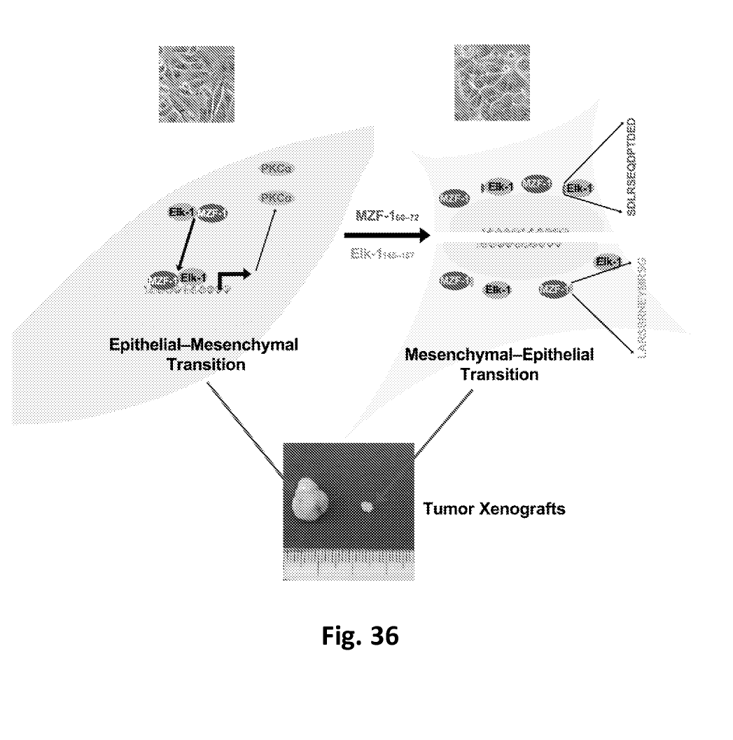

In summary, a Scheme Depicting the Regulation of PKCα Expression by the Cooperation Interaction of Elk-1 and MZF-1 was shown in FIG. 36. MZF-1 and Elk-1 interact in cooperative manner to regulate PKCα expression. Interrupting their interactions by peptides led to decreased DNA binding activity, followed by reduced PKCα expression, eventually attenuating EMT potential and tumorigenesis. Blocking of Elk-1/MZF-1 interaction through the saturation of Elk-1 or MZF-1 binding domains, such as through the application of cell-penetrating HIV transactivating regulatory protein-fused peptides is a therapeutic strategy in the treatment of cancer cells.

What is claimed is:

1. A pharmaceutical composition for inhibiting interaction between MZF-1 and Elk-1, wherein said pharmaceutical composition comprises at least one selected from the group consisting of
(A) Peptides of MZF-1₆₀₋₇₂ having at least 50% sequence identity thereto SEQ ID NO:65, with variations except in the 1^{st}, 3^{rd}, 6^{th}, and 12^{th} amino acid (Aspartic acid, Asp, D);
(B) Peptides of Elk-1₁₄₅₋₁₅₇ having at least 50% sequence identity thereto SEQ ID NO:66, with variations except in the 3^{rd}, 6^{th}, and 11^{th} amino acid (Arginine, Arg, D);
(C) TAT-fused peptide MZF-1₆₀₋₇₂; and
(D) TAT-fused peptide Elk-1₁₄₅₋₁₅₇.

2. The pharmaceutical composition of claim 1, wherein said TAT-fused peptide MZF-1₆₀₋₇₂ sequence is set forth in SEQ ID NO: 57.

3. The pharmaceutical composition of claim 1, wherein said TAT-fused peptide Elk-1₁₄₅₋₁₅₇ sequence is set forth in SEQ ID NO: 59.

4. The pharmaceutical composition of claim 1, further comprises pharmaceutically acceptable vehicles, wherein said vehicles include excipients, diluents, thickeners, fillers, binders, disintegrants, lubricants, oil or non-oil agents, surfactants, suspending agents, gelling agents, adjuvants, preservatives, antioxidants, stabilizers, coloring agents, or spices thereof.

5. The pharmaceutical composition of claim 1, wherein the pharmaceutical composition is given by oral administration, immersion, injection, topical application, or patch administration.

6. A method for treatment of a patient having a cancer, said method comprising administering to the patient a therapeutically effective amount of at least one selected from the group consisting of
(A) Peptides of MZF-1₆₀₋₇₂, the sequence as set forth in SEQ ID NO:65;
(B) Peptide Elk-1₁₄₅₋₁₅₇, the sequence as set forth in SEQ ID NO:66;
(C) Peptides of MZF-1₆₀₋₇₂ having at least 50% sequence identity thereto SEQ ID NO:65, with variations except in the 1^{st}, 3^{rd}, 6^{th}, and 12^{th} amino acid (Aspartic acid, Asp, D)
(D) Peptides of Elk-1₁₄₅₋₁₅₇ having at least 50% sequence identity thereto SEQ ID NO:66, with variations except in the 3^{rd}, 6^{th}, and 11^{th} amino acid (Arginine, Arg, D).
(E) TAT-fused peptide MZF-1₆₀₋₇₂; and
(F) TAT-fused peptide Elk-1₁₄₅₋₁₅₇;

7. The method of claim 6, wherein said TAT-fused peptide MZF-1₆₀₋₇₂ sequence as set forth in SEQ ID NO: 57.

8. The method of claim 6, wherein said TAT-fused peptide Elk-1₁₄₅₋₁₅₇ sequence as set forth in SEQ ID NO: 59.

9. The method of claim 6, wherein said method inhibits interaction between MZF-1 and Elk-1.

10. The method of claim 6, wherein said method reduces tumor volume.

11. The method of claim 6, wherein said cancer more preferably breast cancers, liver cancers or cancers expressing interaction between MZF-1 and Elk-1.

12. The method of claim 11, wherein said breast cancer is triple-negative breast cancer.


SEQUENCE LISTING (66 sequences)

SEQ ID NO: 1 - DNA, 27 bp, Homo sapiens
ggaattccaatgaatggtccccttgtg

SEQ ID NO: 2 - DNA, 26 bp, Homo sapiens
ggggtaccctcggcgctgtggacgcg

SEQ ID NO: 3 - DNA, 27 bp, Homo sapiens
ggaattccaatgaatggtccccttgtg

SEQ ID NO: 4 - DNA, 30 bp, Homo sapiens
cccaagcttggggctggggagcagaacgcg

SEQ ID NO: 5 - DNA, 27 bp, Homo sapiens
ggaattccaatgaatggtccccttgtg

SEQ ID NO: 6 - DNA, 29 bp, Homo sapiens
ggggtacccccatcctcgtccgtggggtc

SEQ ID NO: 7 - DNA, 27 bp, Homo sapiens
ggaattccaatgaatggtccccttgtg

SEQ ID NO: 8 - DNA, 27 bp, Homo sapiens
ggggtacccccgccgcactcgctgcac

SEQ ID NO: 9 - DNA, 30 bp, Homo sapiens
ggaattccaccctgccggggtgtgggccct

SEQ ID NO: 10 - DNA, 29 bp, Homo sapiens
ggggtacccccctcggcgctgtggacgcg

SEQ ID NO: 11 - DNA, 60 bp, Homo sapiens
ggaattccaagcgatctgaggagtgaacaggaccccacggacgaggatcccaagcttggg

SEQ ID NO: 12 - DNA, 60 bp, Homo sapiens
cccaagcttgggatcctcgtccgtggggtcctgttcactcctcagatcgcttggaattcc

SEQ ID NO: 13 - DNA, 60 bp, Homo sapiens
ggaattccaagcgcactgaggagtgaacaggcacccacggcagaggcacccaagcttggg

SEQ ID NO: 14 - DNA, 60 bp, Homo sapiens
cccaagcttgggtgcctctgccgtgggtgcctgttcactcctcagtgcgcttggaattcc

SEQ ID NO: 15 - DNA, 27 bp, Homo sapiens
ggaattccaatgaatggtccccttgtg

SEQ ID NO: 16 - DNA, 51 bp, Homo sapiens
cccaagcttgggtgcctctgccgtgggtgcctgttcactcctcagtgcgct

SEQ ID NO: 17 - DNA, 26 bp, Homo sapiens
ggaattcaatgaatggtccccttgtg

SEQ ID NO: 18 - DNA, 27 bp, Homo sapiens
ccgctcgagctcggcgctgtggacgcg

SEQ ID NO: 19 - DNA, 26 bp, Homo sapiens
ggaattcaatgaatggtccccttgtg

SEQ ID NO: 20 - DNA, 30 bp, Homo sapiens
ccgctcgagcccatcctcgtccgtggggtc

SEQ ID NO: 21 - DNA, 26 bp, Homo sapiens
cgggatccatggacccatctgtgacg

SEQ ID NO: 22 - DNA, 27 bp, Homo sapiens
cccaagctttggcttctggggccctgg

SEQ ID NO: 23 - DNA, 26 bp, Homo sapiens
cgggatccatggacccatctgtgacg

SEQ ID NO: 24 - DNA, 27 bp, Homo sapiens
cccaagcttcacaaacttgtagacgaa

SEQ ID NO: 25 - DNA, 27 bp, Homo sapiens
ggaattccatcctaccctgaggtcgct

SEQ ID NO: 26 - DNA, 27 bp, Homo sapiens
cccaagcttaccgcctgggcctgccat

SEQ ID NO: 27 - DNA, 27 bp, Homo sapiens
ggaattccatcctaccctgaggtcgct

SEQ ID NO: 28 - DNA, 27 bp, Homo sapiens
cccaagcttcgggctgagtggaagctc

SEQ ID NO: 29 - DNA, 27 bp, Homo sapiens
ggaattccatcctaccctgaggtcgct

SEQ ID NO: 30 - DNA, 27 bp, Homo sapiens
cccaagctttggcttctggggccctgg

SEQ ID NO: 31 - DNA, 27 bp, Homo sapiens
ggaattccacttccactcagcccgagc

SEQ ID NO: 32 - DNA, 27 bp, Homo sapiens
cccaagctttggcttctggggccctgg

SEQ ID NO: 33 - DNA, 26 bp, Homo sapiens
cgggatccttggcacgcagcagccgg

SEQ ID NO: 34 - DNA, 27 bp, Homo sapiens
cccaagctttggcttctggggccctgg

SEQ ID NO: 35 - DNA, 57 bp, Homo sapiens
ggaattccattggcacgcagcagccggaacgagtacatgcgctcgggcaagcttggg

SEQ ID NO: 36 - DNA, 57 bp, Homo sapiens
cccaagcttgcccgagcgcatgtactcgttccggctgctgcgtgccaatggaattcc

SEQ ID NO: 37 - DNA, 54 bp, Homo sapiens
ggaattccacagccgcagaagggccggaagccccgggacctagagaagcttggg

SEQ ID NO: 38 - DNA, 54 bp, Homo sapiens
cccaagcttctctaggtcccggggcttccggcccttctgcggctgtggaattcc

SEQ ID NO: 39 - DNA, 57 bp, Homo sapiens
ggaattccattggcagcaagcagcgcaaacgagtacatggcatcgggcaagcttggg

SEQ ID NO: 40 - DNA, 57 bp, Homo sapiens
cccaagcttgcccgatgccatgtactcgtttgcgctgcttgctgccaatggaattcc

SEQ ID NO: 41 - DNA, 54 bp, Homo sapiens
ggaattccattggcagcaagcagcgcaaacgagtacatggcatcgggcctctat

SEQ ID NO: 42 - DNA, 27 bp, Homo sapiens
cccaagctttggcttctggggccctgg

SEQ ID NO: 43 - DNA, 30 bp, Homo sapiens
ggggtaccccaaatggacccatctgtgacg

SEQ ID NO: 44 - DNA, 27 bp, Homo sapiens
cgggatcccgtcatggcttctggggcc

SEQ ID NO: 45 - DNA, 30 bp, Homo sapiens
ggaattccaatggctgacgttttcccgggc

SEQ ID NO: 46 - DNA, 30 bp, Homo sapiens
cccaagctttactgcactctgtaagatggg

SEQ ID NO: 47 - DNA, 20 bp, Homo sapiens
ggtacaggcagctaaaacac

SEQ ID NO: 48 - DNA, 19 bp, Homo sapiens
gtcttccttctcccactcc

SEQ ID NO: 49 - DNA, 34 bp, Homo sapiens
cctgaggatggggaaggggcttcctgctgcggtg

SEQ ID NO: 50 - DNA, 34 bp, Homo sapiens
caccgcagcaggaagccccttccccatcctcagg

SEQ ID NO: 51 - DNA, 34 bp, Homo sapiens
cctgcgtatttttaaggggcttcctgctgcggtg

SEQ ID NO: 52 - DNA, 34 bp, Homo sapiens
caccgcagcaggaagccccttaaaaatacgcagg

SEQ ID NO: 53 - DNA, 34 bp, Homo sapiens
cctgcggatggggaaggggattaatgatgaggtg

SEQ ID NO: 54 - DNA, 34 bp, Homo sapiens
cacctcatcattaatccccttccccatccgcagg

SEQ ID NO: 55 - DNA, 34 bp, Homo sapiens
cctgcgtatttttaaggggattaatgatgaggtg

SEQ ID NO: 56 - DNA, 34 bp, Homo sapiens
cacctcatcattaatccccttaaaaatacgcagg

SEQ ID NO: 57 - protein, 27 aa, Homo sapiens
YGRKKRRQRRRGGGDEDTPDQESRLDS

SEQ ID NO: 58 - protein, 27 aa, Homo sapiens
YGRKKRRQRRRGGGAEATPAQESRLAS

SEQ ID NO: 59 - protein, 27 aa, Homo sapiens
YGRKKRRQRRRGGGLARSSRNEYMRSG

SEQ ID NO: 60 - protein, 27 aa, Homo sapiens
YGRKKRRQRRRGGGLAASSANEYMASG

SEQ ID NO: 61 - DNA, 21 bp, Homo sapiens
cagcgtcacagatgggtccat

SEQ ID NO: 62 - DNA, 21 bp, Homo sapiens
atggacccatctgtgacgctg

SEQ ID NO: 63 - DNA, 22 bp, Homo sapiens
tacacaaggggaccattcattc

SEQ ID NO: 64 - DNA, 22 bp, Homo sapiens
gaatgaatggtccccttgtgta

SEQ ID NO: 65 - protein, 13 aa, Homo sapiens
DEDTPDQESRLDS

SEQ ID NO: 66 - protein, 13 aa, Homo sapiens
LARSSRNEYMRSG